(19) United States
(12) Patent Application Publication
(10) Pub. No.: US 2003/0157583 A1
(43) Pub. Date: Aug. 21, 2003

(54) Methods for determining squalene synthase activity

(76) Inventors: Donna Stevens, Hillsborough, NC (US); Xiao-Zhuo Wang, East Lyme, CT (US); John Rice, Pittsboro, NC (US); Beth Nye, Morrisville, NC (US); David Broadwell, Garner, NC (US); Norman Glassbrook, Chapel Hill, NC (US); Veeresh Sevala, Cary, NC (US); John Crawford, Raleigh, NC (US); Sandy Stewart, Durham, NC (US)

Correspondence Address: PARADIGM GENETICS, INC, 108 ALEXANDER DRIVE, P O BOX 14528, RTP, NC, 27709-4528, US

(21) Appl. No.: 10/024,130

(22) Filed: Dec. 17, 2001

(51) Int. Cl. C12Q 1/48; C07H 21/04; C12Q 1/26; C12N 9/02; C12N 9/10; C12P 21/02; C12N 5/04

(52) U.S. Cl. 435/15; 435/193; 435/25; 435/69.1; 435/189; 435/320.1; 435/419; 536/23.2

(57) ABSTRACT

The cloning of a truncated Arabidopsis gene expressing squalene synthase, as well as the expression and purification of the squalene synthase, are described. Also described herein is a fluorescent assay using squalene synthase that is amenable to high-throughout use, particularly for studying the regulation of isoprenoid synthesis and identifying squalene synthase inhibitors and promoters. As the formation of squalene is stoichiometric with the depletion of NADPH, the activity of squalene synthase can be evaluated by following the NADPH concentration over time. Squalene synthase activity is determined by combining FPP, NADPH, squalene synthase and a magnesium ion cofactor to form a reaction mixture under conditions suitable for squalene formation, optionally in the presence of a compound being analyzed for its ability to inhibit or promote squalene synthase. The concentration of NADPH over time is determined by subjecting the reaction mixture to UV light and detecting fluorescent light emission.

### FIELD OF THE INVENTION

The invention relates generally to assays for determining squalene concentration and squalene synthase activity.

### BACKGROUND OF THE INVENTION

Squalene synthase [E.C.2.5.1.21] is the first pathway-specific enzyme in sterol biosynthesis (Zhang et al., Arch. of Biochem. and Biophys., 304(1):133-143 (1993)). A bifunctional enzyme, it catalyzes the conversion of two molecules of famesyl diphosphate (FPP) into an intermediate, presqualene diphosphate (PSPP), followed by the conversion of presqualene diphosphate into squalene in the presence of NADPH and magnesium, as shown below in Scheme I.

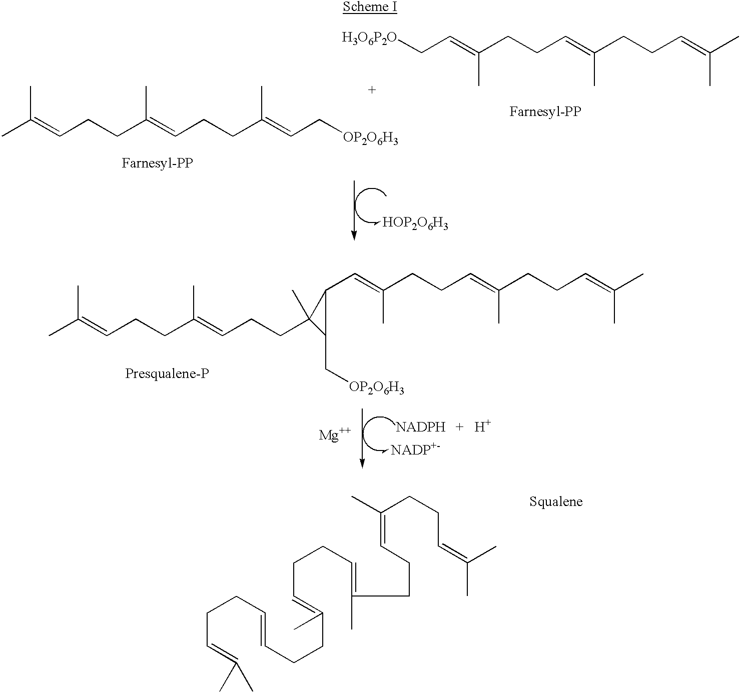

### Scheme I

The substrate FPP lies at the branch point in the isoprenoid biosynthetic pathway, functioning as a metabolic intermediate in the formation of dolichols, ubiquinones, cholesterol, isoprenoids, and farnesylated proteins. Squalene synthase is a microsomal protein, with its C-terminal hydrophobic residues anchoring the enzyme to the endoplasmic reticulum membrane. The enzyme is noted for its connection of the cytosolic and microsomal segments of sterol biosynthesis, converting a hydrophilic protein to one that is hydrophobic. Squalene synthase has been reported to be resistant to solubilization and purification (Soltis et al., Arch. Biochem. and Biophys. 316(2):713-723 (1995)).

Squalene is an intermediate in cholesterol and steroid biosynthesis. It is formed from presqualene pyrophosphate in the walls of the endoplasmic reticulum using electrons from NADPH. In the reaction, the pyrophosphate is removed from the molecule. Subsequently, squalene is cyclized to lanosterol, which is subsequently converted to cholesterol. Cholesterol is ubiquitous in eukaryotes but absent from most prokaryotes.

In humans and other animals, sterols and their derivatives are essential metabolites related to the endocrine system and immune system, and are important for regulating cell membrane processes. Cholesterol and its fatty acyl esters are important structural components of membranes. Cholesterol also serves as precursor for the synthesis of steroid hormones, vitamin D, and bile salts.

Steroid hormones are used for a broad range of signaling mechanisms. Cholesterol is a precursor to pregnenolone, progestagens, androgens and estrogens (the male and female sex hormones), mineral corticoids such as aldosterone (used to control kidney function), and glucocorticoids such as cortisol, which are activators of gluconeogenesis, glycogen formation, and fat and protein degradation. Bile acids are hydrophilic cholesterol derivatives. They are synthesized in the liver and stored in the gallbladder, where they are released into the small intestine to help solubilize dietary fats.

In plants, squalene is converted to squalene epoxide, which is then cyclized to form cycloartenol. Cycloartenol is formed in an early stage in the biosynthetic pathway of sterol production in higher plants. Squalene epoxide can also be converted into pentacyclic sterols, containing five instead of four rings. Exemplary pentacyclic sterols include the phytoalexins and saponins. Several plant squalene synthase genes have been cloned, including daffodil and Arabidopsis thaliana (Scolnik and Bartley, Plant Molecular Biology Reporter, 14 (4): 305-319 (1996), accession number xb6692, Kribii et al., “Molecular cloning, expression and characterization of cDNAs for Arabidopsis thaliana squalene synthase” (1995). Direct Submission. Unpublished.)

Cycloartenol is one of the first sterols in the higher plant biosynthetic pathway, and is a precursor numerous other sterols. Examples of naturally occurring delta-5 plant sterols include isofucosterol, sitosterol, stigmasterol, campesterol, cholesterol, and dihydrobrassicasterol. Examples of naturally occurring non-delta-5 plant sterols include cycloartenol, 24-methylene cycloartenol, cycloeucalenol, and obtusifoliol.

Insects are unable to synthesize de novo the steroid nucleus and depend upon external sources of sterols in their food source for production of necessary steroid compounds. In particular, insect pests require an external source of delta-5 sterols, particularly to form ecdysteroids, hormones that control insect reproduction and development (Costet et al., Proc. Natl. Acad. Sci. USA, 84:643 (1987) and Corio-Costet et al., Archives of Insect Biochem. Physiol., 11:47 (1989)). The ratio of delta-S to non-delta-5 sterols in plants is an important factor relating to insect pest resistance.

Yeasts such as Leishmania major also have a squalene synthase gene. The complete code for the Leishmania major squalene synthase gene, as well as the protein sequence for the squalene synthase, is available from GenBank. Various fungi also have a squalene synthase gene, and inhibitors of fungal squalene synthase can be active as antifungal agents.

The zaragozic acids are very potent inhibitors of squalene synthase that inhibit cholesterol synthesis and lower plasma cholesterol levels in primates (Bergstrom et al., Proc. Natl. Acad. Sci. USA 90, 80-84 (1993)). They also inhibit fungal ergosterol synthesis and are potent fungicidal compounds. Squalene synthase inhibitors have potential as cholesterol lowering agents and/or as antifungal agents (Ciosek et al.,. J. Biol. Chem., 269(33):24832 (1993)).

The prior art assays for squalene synthase activity generally involved using radiolableled FPP directly measuring degradations over time. However, this type of assay is not readily adaptable to high throughput screening assays.

Accordingly, it would be advantageous to develop purified squalene synthase that can be used in high throughput assays, as well as high throughput assays for squalene synthase activity. The present invention provides such assays and purified squalene synthase.

### SUMMARY OF THE INVENTION

The cloning of a truncated Arabidopsis gene expressing squalene synthase, as well as the expression and purification of the squalene synthase, are described herein. Also described herein is a fluorescent assay using squalene synthase that is amenable to high-throughout use, particularly for studying the regulation of isoprenoid synthesis and identifying squalene synthase promoters and inhibitors.

Assays for determining squalene synthase activity and methods for identifying agents that promote or inhibit squalene synthase activity are described. Squalene synthase inhibitors can be used, for example, as herbicides, fungicides or insecticides, to lower cholesterol levels in humans and other animals, and to control isoprenoid biosynthetic pathways in humans and other animals.

Squalene synthase activity can be determined by combining FPP, NADPH, squalene synthase and a magnesium ion cofactor to form a reaction mixture under conditions suitable for squalene formation, optionally in the presence of a compound being analyzed for its ability to inhibit or promote squalene synthase activity.

Squalene formation is stoichiometric with NADPH depletion, so the activity of squalene synthase can be evaluated by following the NADPH concentration over time. The concentration of NADPH over time is determined by subjecting the reaction mixture to UV light and detecting fluorescent light emission.

Methods for identifying test compounds that function as squalene synthase promoters or inhibitors involve combining FPP, NADPH, a magnesium ion cofactor and a suitable plant, fungal or animal squalene synthase to form a reaction mixture in the presence and absence of the test compound. The reaction mixture is exposed to UV light while the reaction is allowed to take place, and the amount of fluorescent light emission is measured. The amount of the fluorescent light emission in the presence and absence of the test compound is compared. A decrease in the amount of the fluorescent light emission over time (hence, an increase in NADPH utilization) in the presence of the test compound indicates that the test compound is a squalene synthase promoter. An increase in the amount of the fluorescent light emission over time (hence, a decrease in NADPH utilization) in the presence of the test compound indicates that the test compound is a squalene synthase inhibitor.

### BRIEF DESCRIPTION OF THE FIGURES

FIG. 1 is a western blot of squalene synthase derived from E. coli, as shown in Example 1, where lane 1 represents the whole cell lysate, lane 2 represents the clarified lysate, lane 3 represents the column flow through, and lane 4 represents elution.

FIG. 2 represents a fluorescence assay comparing fluorescence (in relative fluorescence units (RFU) measured at 340 nm excitation/465 nm emission) and micrograms truncated squalene synthase (tSqS) where farnesyl diphosphate (FPP) was converted by squalene in the presence of NADPH, as shown in Example 2.

FIGS. 3A-3F represent GC/MS spectra resulting from the conversion of FPP to squalene in the presence of NAPDH and tSqS. 3a represents a solvent blank. 3b represents an extraction blank. 3c and d represent reactions including tSqS. 3e represents a reaction fortified with squalene. 3f represents a squalene standard.

FIG. 4 represents a titration of tSqS into a substrate containing FPP and NADPH, incubated for 30 minutes at 37° C., in terms of fluorescence (RFU) versus micrograms tSqS.

FIG. 5 shows the decrease in fluorescence (RFU) over time (minutes) as NADPH is used to convert FPP to squalene in the presence of varying concentrations of tSqS.

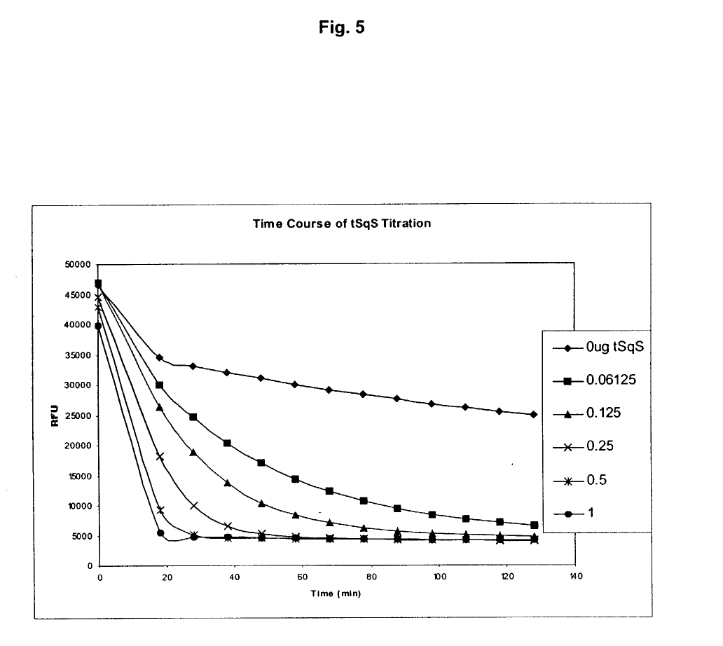

FIG. 6 the determination of the Km for FPP, in terms of initial velocity (Vo) versus concentration of FPP (μM).

FIG. 7 is a bar graph showing the fluorescence (RFU) versus NAPDH concentration (μM) where no enzyme was present (bracketed bars), 125 ng tSqS was present (dark bars) and where no NADPH was present (empty bars).

FIG. 8 is a bar graph showing the optimization of the magnesium ion cofactor in the conversion of FPP to squalene using tSqS, in terms of RFU versus mM magnesium chloride (MgCl₂).

FIG. 9 is a graph showing the effect of temperature on squalene synthase activity, in terms of fluorescence (RFU) versus time (min), where the circles represent results obtained at room temperature and the squares represent results obtained at 37° C.

FIG. 10 is a graph representing the effect of a five minute pre-incubation of the substrate (NADPH, FPP and MgCl₂) with tSqS. The bracketed bars represent results where no enzyme was added, the darkened bars represent results where 100 ng of tSqS was added, and the light bars represent the difference between these two values.

FIG. 11 is graph comparing the fluorescence (RFU) over time where no tSqS was added (diamonds) and 125 ng tSqS was added (squares) to a mixture of FPP and NADPH.

FIG. 12 is a graph comparing the fluorescence (RFU) over time (min) for the conversion of FPP to squalene in the presence of NADPH using three different lots of tSqS.

FIG. 13 is a bar graph comparing the fluorescence (RFU) versus bovine serum albumin concentration (mg/ml) in the conversion of FPP to squalene in the presence of NADPH using tSqS. Bracketed bars show results where no enzyme was added, and darkened bars show results where 125 ng of tSqS was added.

FIG. 14 is a graph showing the stability of FPP when stored at 4° C. as determined by converting the FPP to squalene with tSqS in the presence of NADPH, as measured in terms of fluorescence (RFU) versus storage time (hours).

FIG. 15 is a graph showing the stability of FPP when stored at room temperature as determined by converting the FPP to squalene with tSqS in the presence of NADPH, as measured in terms of fluorescence (RFU) versus storage time (hours).

FIG. 16 is a graph showing the stability of tSqS when stored at 4° C. as determined by converting FPP to squalene with the tSqS in the presence of NADPH, as measured in terms of fluorescence (RFU) versus storage time (hours).

FIG. 17 is a graph showing the stability of tSqS when stored at 4° C. as determined by converting FPP to squalene with the tSqS in the presence of NADPH, as measured in terms of fluorescence (RFU) versus storage time (hours).

FIG. 18 is a graph showing the stability of tSqS when stored at various temperatures in the presence of varying amounts of glycerol as determined by converting FPP to squalene with the tSqS in the presence of NADPH, as measured in terms of fluorescence (RFU).

FIG. 19 is a graph showing the stability of NAPDH when stored at 4° C. as measured in terms of fluorescence (RFU) versus storage time (hours).

FIG. 20 is a graph showing the stability of tSqS when incubated at 37° C. as measured in terms of fluorescence (RFU) versus incubation time (min).

FIG. 21 is a graph showing the effect of varying concentrations of DMSO on the effectiveness of tSqS as determined by converting FPP to squalene with the tSqS in the presence of NADPH, in terms of fluorescence (RFU) versus DMSO (vol. %), where the bracketed bars show the results where no tSqS was present, and the darkened bars show the results where 125 ng of tSqS was present.

FIG. 22 is a graph showing the inhibition of squalene synthesis by EDTA chelation of the Mg⁺⁺ cofactor with EDTA, as measured by fluorescence (RFU) versus EDTA concentration (mM).

FIG. 23 is a scatterplot graph showing the results of a high throughput (384-well plate) squalene synthase assay, in terms of RFU versus well number. The diamonds represent results obtained with fresh reagents and no tSqS added. The squares represent results obtained with fresh reagents and tSqS added. The triangles represent results obtained with reagents aged for approximately 24 hours at 4° C. with no tSqS added and the x's represent results obtained with aged reagents with tSqS added.

FIG. 24 is a scatterplot graph showing the results of a high throughput (384-well plate) squalene synthase assay, in terms of RFU versus well number. The diamonds represent results obtained using an opaque plate, with no tSqS added. The squares represent results obtained using an opaque plate, and tSqS added. The triangles represent results obtained with a clear bottom plate, with no tSqS added and the x's represent results obtained with a clear bottom plate, with tSqS added.

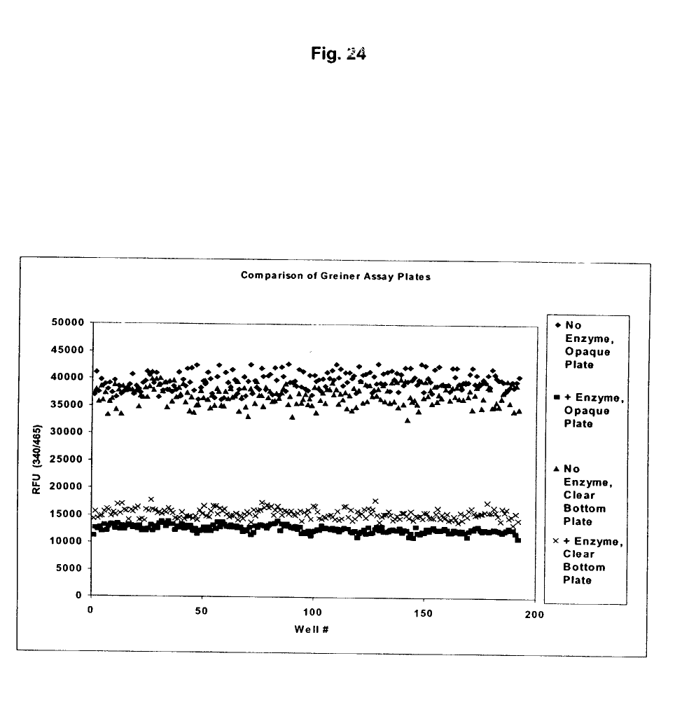

### DETAILED DESCRIPTION OF THE INVENTION

Assay methods for determining squalene synthase activity and identifying squalene synthase inhibitors and/or promoters are described. The assays are particularly suited to high throughput assay procedures. The assays are based on the detection of NADPH, which is stoichiometrically converted to NADP during the conversion of famesyl disphosphate (FPP) to squalene in the presence of squalene synthase and a magnesium ion cofactor.

Recombinant truncated squalene synthase from Arabidopsis thaliana, an example of which is shown in SEQ ID NO: 6, and squalene synthase proteins with one or more conservative changes, compared with the amino acid sequence of SEQ ID NO: 6 are also disclosed. Nucleic acid molecules that encode a truncated squalene synthase polypeptide as described in SEQ ID NO:. 6, as well as nucleic acid molecules that encode proteins having one or more conservative amino acid changes, compared with the amino acid sequence of SEQ ID NO: 6 are also disclosed. The present invention thus includes polypeptides that include a sequence that is at least about 50%, preferably at least 60% or 70%, and more preferably 80%, 85%, 90%, 95%, or 98% identical to the amino acid sequence of SEQ ID NO: 6.

NADPH gives off fluorescence whereas NADP gives off significantly less fluorescence when exposed to UV radiation, for example, at 340 nm. This allows the indirect measurement of squalene synthesis by following the loss of NADPH over time.

The following definitions will be useful in understanding the methods and assays described herein.

Definitions

Squalene is an intermediate in cholesterol and steroid biosynthesis. It is formed from presqualene pyrophosphate in the walls of the endoplasmic reticulum using electrons from NADPH.

As used herein, the term “squalene synthase” (EC.2.5.1.21) refers to any enzyme that catalyzes the formation of squalene from FPP in the presence of NADPH and a magnesium ion cofactor.

Farnesyl diphosphate (FPP) is an isoprenoid with the formula C₁₅H₂₈O₇P₂, with the structure shown in Scheme 1. Farnesyl diphosphate is the immediate precursor of squalene, which it forms by undergoing tail-to-tail condensation in the presence of squalene synthase under anaerobic conditions. Two molecules of FPP are reacted with squalene synthase to form presqualene diphosphate, which is reacted with NADPH to form squalene.

Nicotinamide adenine dinucleotide phosphate (NADP⁺) is an important coenzyme, functioning as a hydrogen and electron carrier in a wide range of redox reactions, including squalene synthesis. The oxidized form of the coenzyme is written NADP⁺ and the reduced form is written as NADPH. NADPH has the formula C₂₁H₃₀N₇O₁₇P₃

Ultraviolet light (UV) is radiant energy below the visible range, typically in the range of about 190-400 nanometers (nm).

The sequence identity within mammalian species is reported to be 90% identical, and 44.8% identical between rat liver and yeast, but very poor in comparison to the Arabidopsis sequence. There appear to be 3 sections (A, B, C) which are involved in the formation of squalene. Section A contains a Tyr residue essential for catalysis, section B contains aspartate-rich regions thought to be involved in the Mg⁺⁺ -salt bridges, and section C contains a unique Phe residue possibly involved in the second step of catalysis (the reduction by NADPH to form squalene). (Gu et al. J. Biol. Chem., 273(20):12515-12525 (1998))

The truncated enzyme used in the working examples described herein was derived from Arabidopsis thaliana. This enzyme is referred to herein as tSqS. Squalene synthase is a bifunctional enzyme which catalyzes the conversion of two molecules of farnesyl diphosphate (FPP) into an intermediate, presqualene diphosphate (PSPP) and also converts PSPP to squalene in the presence of NADPH and magnesium ions. Other enzymes that produce squalene from FPP or PSPP in the presence of NADPH are also contemplated for use in the assay methods.

The term “herbicide,” as used herein, refers to a compound that may be used to kill or suppress the growth of at least one plant, plant cell, plant tissue or seed.

The term “fungicide,” as used herein, refers to a compound that may be used to kill or suppress the growth of at least one fungus.

The term “inhibitor,” as used herein, refers to a chemical substance that wholly or partially inactivates the enzymatic activity of squalene synthase The inhibitor may function by interacting directly with the enzyme, a co-factor of the enzyme, the substrate of the enzyme, or any combination thereof.

The tern “promoter,” as used herein, refers to a chemical substance that increases the enzymatic activity of squalene synthase. The promoter may function by interacting directly with the enzyme, a co-factor of the enzyme, the substrate of the enzyme, or any combination thereof.

The term “squalene synthase inhibitor,” as used herein, refers to a compound that inhibits squalene formation catalyzed by squalene synthase.

The term “squalene synthase promoter,” as used herein, refers to a compound that promotes squalene formation catalyzed by squalene synthase.

The term “insecticide,” as used herein, refers to a compound that may be used to kill or suppress the growth of at least one insect.

The term “selective fungicide,” as used herein, refers to a compound that may be used to kill or suppress the growth of at least one fungus while not significantly adversely affecting a plant, plant cell, plant tissue or seed.

The term “selective insecticide,” as used herein, refers to a compound that may be used to kill or suppress the growth of at least one insect while not significantly adversely affecting a plant, plant cell, plant tissue or seed.

The term “conservative amino acid substution” refers to a substitution represented by a BLOSUM62 value of greater than −1. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. For example, an amino acid substitution is conservative if the substitution is characterized by a BLOSUM62 value of 0, 1, 2, or 3. Preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Variant squalene synthase polypeptides or substantially homologous squalene synthase polypeptides are characterized as having one or more amino acid substitutions, deletions or additions. These changes are preferably of a minor nature, that is conservative amino acid substitutions (see Table 1) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

TABLE 1
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The “percent (%) sequence identity” between two polynucleotide or two polypeptide sequences is determined according to the either the BLAST program (Basic Local Alignment Search Tool; Altschul and Gish, Meth. Enzymol., 266:460-480 (1996) and Altschul, J. Mol. Biol., 215:403-410 (1990)) in the Wisconsin Genetics Software Package (Devererreux et al., Nucl. Acid Res. 12:387 ((1984)), Genetics Computer Group (GCG), Madison, Wis. (NCBI, Version 2.0.11, default settings) or using Smith Waterman Alignment (Smith and Waterman, Adv. AppL Math. 2:482 (1981)) as incorporated into GeneMatcher Plus™ (Paracel, Inc., http://www.paracel.com/html/genematcher.html; using the default settings and the version current at the time of filing). It is understood that for the purposes of determining sequence identity when comparing a DNA sequence to an RNA sequence, a thymine nucleotide is equivalent to a uracil nucleotide.

The terms “fungus,” “fungi,” “fungal pathogen” or “fungal phytopathogen” as used herein refer to species of the taxonomic group Myceteae and which are capable of pathogenically infecting plants or animals. For example, fungal phytopathogens include, but are not limited to, Alternaria spp., Aspergillus spp., including As. nidulans, Botrytis spp., Ceratocystis spp., Fusarium spp. including F. oxysporum, and F. roseum, Helminthosporum spp., Hemileia spp., Lasiodiplodia theobromae, Magnaporthe grisea, Meliola spp., Mucor spp., Mycosphaerella spp. including M. graminicola, Neurospora spp. including N. crassa, Oidium spp., Phoma spp., Phyllosticta spp., Sclerotina spp., Septoria spp., Trichoderma spp., Uromyces spp. and Verticillium spp. Fungal pathogens of animals and humans include, but are not limited to, Aspergillus spp., Nocardia spp., Penicillum spp., Rhizopus spp., Mucor spp., Blastomyces dermatitidis, Candida spp. including C. albicans, Saccharomyces spp., Trichosporon spp., and Trichophyton spp. The term “pathogen” as used herein refers to an organism such as a fungus, a bacterium or protozoan capable of producing a disease in a plant or animal. The term “phytopathogen” as used herein refers to a pathogenic organism that infects a plant.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

The term “selectively inhibiting” refers to inhibiting the squalene synthase activity of a pathogen to a different degree than that of a host of the pathogen. The term “selectively inhibiting” can further refer to inhibiting the proliferation of a pathogen such as, but not limited to, a fingal phytopathogen whereas the proliferation of the pathogen host is not significantly inhibited.

As used herein the terms “polypeptide” and “protein” refer to a polymer of amino acids of three or more amino acids, preferably four or more amino acids, in a serial array, linked through peptide bonds. The chain may be linear, branched, circular or combinations thereof. The polypeptides may contain amino acid analogs and other modifications, including, but not limited to glycosylated or phosphorylated residues.

The term “polypeptide” includes proteins, protein fragments, protein analogues, oligopeptides and the like. The term “polypeptides” contemplates polypeptides as defined above that are encoded by nucleic acids, produced through recombinant technology, isolated or purified from an appropriate source such as a plant or fungus, or are synthesized. The term “polypeptides” further contemplates polypeptides as defined above that include chemically modified amino acids or amino acids covalently or noncovalently linked to labeling ligands.

The term “specific binding” refers to an interaction between squalene synthase and a molecule or compound, wherein the interaction is dependent upon the primary amino acid sequence or the conformation of squalene synthase.

As used herein, “magnesium” refers to any suitable magnesium ion useful as a cofactor for the squalene synthase. Examples of magnesium ions useful in the assay methods described herein include, but are not limited to, magnesium chloride, magnesium sulfate and the like.

The term “nucleic acid” as used herein refers to any natural or synthetic linear and sequential arrays of nucleotides and nucleosides, for example cDNA, genomic DNA, mRNA, tRNA, oligonucleotides, oligonucleosides and derivatives thereof. For ease of discussion, such nucleic acids can be collectively referred to herein as “constructs,” “plasmids,” or “vectors.” The term “nucleic acid” further includes modified or derivatized nucleotides and nucleosides such as, but not limited to, halogenated nucleotides such as, but not only, 5-bromouracil, and derivatized nucleotides such as biotin-labeled nucleotides.

The term “isolated nucleic acid” as used herein refers to a nucleic acid with a structure (a) not identical to that of any naturally occurring nucleic acid or (b) not identical to that of any fragment of a naturally occurring genomic nucleic acid spanning more than three separate genes, and includes DNA, RNA, or derivatives or variants thereof. The term covers, for example, (a) a DNA which has the sequence of part of a naturally occurring genomic molecule but is not flanked by at least one of the coding sequences that flank that part of the molecule in the genome of the species in which it naturally occurs; (b) a nucleic acid incorporated into a vector or into the genomic nucleic acid of a prokaryote or eukaryote in a manner such that the resulting molecule is not identical to any vector or naturally occurring genomic DNA; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, i.e., a gene encoding a fusion protein, and (e) a recombinant nucleotide sequence that is part of a hybrid sequence that is not naturally occurring. Isolated nucleic acid molecules of the present invention can include, for example, natural allelic variants as well as nucleic acid molecules modified by nucleotide deletions, insertions, inversions, or substitutions such that the resulting nucleic acid molecule still essentially encodes an enzyme active in the purine biosynthetic pathway.

It is advantageous for some purposes that a nucleotide sequence or a protein or polypeptide is in purified form. The term “purified” in reference to nucleic acids, proteins or polypeptides represents that the nucleic acid, protein or polypeptide has increased purity relative to the natural environment.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein or polypeptide or a portion thereof.

The term “fragment” as used herein to refer to a nucleic acid (e.g., cDNA) refers to an isolated portion of the subject nucleic acid constructed artificially (e.g., by chemical synthesis) or by cleaving a natural product into multiple pieces, using restriction endonucleases or mechanical shearing, or a portion of a nucleic acid synthesized by PCR, DNA polymerase or any other polymerizing technique well known in the art, or expressed in a host cell by recombinant nucleic acid technology well known to one of skill in the art. The term “fragment” as used herein can also refer to an isolated portion of a polypeptide, wherein the portion of the polypeptide is cleaved from a naturally occurring polypeptide by proteolytic cleavage by at least one protease, or is a portion of the naturally occurring polypeptide synthesized by chemical methods well known to one of skill in the art.

The term “microarray” as used herein refers to an arrangement of distinct polynucleotides, peptides or polypeptides arranged on a substrate, e.g. paper, nylon, any other type of membrane, filter, chip, glass slide, silicone wafer, or any other suitable solid or flexible support.

### I. Assay Components

Squalene Synthase

By “squalene synthase” is meant any enzyme which catalyzes the formation of squalene from FPP or PSPP in the presence of NADPH and a magnesium ion cofactor. Methods for measuring squalene synthase activity are described herein

The squalene synthase may have the amino acid sequence of a naturally occurring squalene synthase found in a plant, fungus, animal or microorganism, or may have an amino acid sequence derived from a naturally occurring sequence. Preferably the squalene synthase is a plant squalene synthase.

By “plant squalene synthase” is meant an enzyme that can be found in at least one plant, and which catalyzes the formation of squalene from FPP or PSPP in the presence of NADPH and a magnesium ion cofactor. The squalene synthase may be from any plant, including both monocots and dicots. In one embodiment, the squalene synthase is an Arabidopsis squalene synthase. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. Preferably, the Arabidopsis squalene synthase is from Arabidopsis thaliana, more preferably from Arabidopsis thaliana strain Columbia.

The cDNAs sequence for the A. thaliana squalene synthase includes 1233 nucleotides and is available in the public domain as accession number X86692.1 (SEQ ID NO: 1). The protein translation of the squalene synthase includes 410 amino acids (SEQ ID NO: 2).

The DNA sequence encoding the squalene synthase C-terminal transmembrane domain, which was excluded from the pET30/tSQS assembly in Example 1, is a 69 nucleotide oligonucleotide (SEQ ID NO: 3), the translation of which is a 22 amino acid peptide (SEQ ID NO: 4). The resulting cDNA encoding the truncated squalene synthase is shown as SEQ ID NO: 5, and the resulting truncated squalene synthase is shown as SEQ ID NO: 6.

In various embodiments, the squalene synthase can be derived from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like. Fragments of a plant squalene synthase may be used in the assays described herein. The fragments comprise at least 10 consecutive amino acids of a plant squalene synthase. Preferably, the fragment comprises at least 15, 20, 25, 30, 35, 40, 50, 60, 70, 80, 90 or at least 100 consecutive amino acids residues of a plant squalene synthase. Most preferably, the fragment comprises at least 10 consecutive amino acid residues of an Arabidopsis squalene synthase. Preferably, the fragment contains an amino acid sequence conserved among plant squalene synthases. Those skilled in the art can identify additional conserved fragments using sequence comparison software.

Polypeptides having at least 80% sequence identity with a plant squalene synthase are also useful in the assay methods described herein. Preferably, the sequence identity is at least 85%, more preferably the identity is at least 90%, most preferably the sequence identity is at least 95%.

In addition, the polypeptide preferably has at least 50% of the activity of a plant squalene synthase. More preferably, the polypeptide has at least 60%, at least 70%, at least 80% or at least 90% of the activity of a plant squalene synthase. Preferably, the activity of the polypeptide is compared to the activity of the truncated Arabidopsis thaliana squalene synthase polypeptide used in the working examples described herein.

Fungal squalene synthases (as well as squalene synthases from those bacteria that include this enzyme) can also be used in the assays. A suitable fungal squalene synthase, for example, that can be the target of a test compound is that of the fungus M. grisea, or derivatives or truncated versions thereof. The yeast squalene synthase derived from Saccharomyces cerevisiae is known in the art and is an example of a yeast squalene synthase that can be used.

Mammalian squalene synthases can also be used, including human squalene synthase and rat squalene synthase. The rat hepatic and human squalene synthases are examples of mammalian squalene synthases whose sequences are known in the art.

With respect to the assays described in the working examples, initial assay development using a partially purified full-length gene was successful (data not shown). However, most of the resulting protein still associated with the pelleted membranes regardless of extraction procedure. Accordingly, this would require that a higher amount of soluble enzyme would be necessary for the screening assay.

The limitations associated with using the entire gene encoding the Arabidopsis thaliana squalene synthase were overcome by clipping off the C-terminal hydrophobic region (SEQ ID NO: 3), which otherwise would have added the peptide sequence in SEQ ID NO: 4 to the resulting squalene synthase, and using the resulting truncated squalene synthase (SEQ ID NO: 6) in the assays. The truncated squalene synthase shown in SEQ ID NO: 6, encoded by the (recombinant) DNA shown in SEQ ID NO: 5, is particularly preferred for use in the assays, although other truncated variants that similarly do not include the C-terminal hydrophobic region are also preferred. The same holds true for squalene synthases derived from other species that encode a sequence for membrane targeting.

Squalene, FPP, NADPH and Mg Ions

Squalene, FPP, NADPH and various sources of magnesium ions, as defined above, are readily available from commercial sources, including, for example, Aldrich Chemicals (St. Louis, Mo.). FPP is available, for example, from Echelon (Salt Lake City, Utah, Item No. 1-0150). NADPH is available, for example, from Sigma (St. Louis, Mo., Item No. N-1630). Magnesium chloride is also available from Sigma (St. Louis, Mo., Item No. M-2670).

Solutions/Media

In those embodiments of the assays that are cell-free assays, any media in which the enzyme is active and in which the reactants and products are soluble can be used. Preferred solutions are buffered solutions, more preferably, solutions buffered to about physiological pH. The solutions can include DMSO or other water-soluble organic solvents that can assist with long term storage of the squalene synthase at reduced temperatures. Examples of suitable aqueous solutions containing DMSO that can be used are described in more detail in the Examples.

In those embodiments of the assays that use whole cells or tissues, any cell culture media capable of sustaining the viability of the cells and also solubilizing the reactants and products can be used. Examples of cell culture media are well known to those of skill in the art.

Compounds

Various types of compounds can be screened for their potential ability to inhibit squalene synthase. Examples include, but are not limited to, enzymes, amino acids and derivatives thereof, proteins (including more than about 70 amino acids), peptides (including between 2 and 70 amino acids), natural and synthetic saccharides, genetic material, viruses, bacteria, vectors and small molecules (molecules with molecular weights less than about 1000).

Compound Libraries

The compounds can be present in combinatorial or other compound libraries, for example, lead generation and/or lead optimization libraries. For purposes of this invention, lead generation libraries are relatively large libraries that contain potential lead compounds, and lead optimization libraries are developed around compounds identified as potential leads by assaying lead generation libraries. Such libraries typically include a large number of compounds, include at least two compounds, and can include upwards of tens of thousands of compounds.

Logically arranged collections of potentially active herbicidal, bactericidal and/or fungicidal compounds can be evaluated using the high throughput bioassays described herein, such that structure-reactivity relationships (SARs) can be obtained. Methods for arranging compounds to be assayed in logical arrangements are known to those of skill in the art, and described, for example, in U.S. Pat. No. 5,962,736 to Zambias et al., the contents of which are hereby incorporated by reference. In one embodiment, the compounds are added to multi-well plates in the form of an “array,” which is defined herein as a logical positional ordering of compounds in Cartesian coordinates, where the array includes compounds with a similar core structure and varying substitutions.

By placing the compounds in a logical array in multi-tube arrays or multi-well plates, the herbicidal, bactericidal or fimgicidal effect of individual compounds can be evaluated, and compared to that of structurally similar compounds to generate SAR data.

Relational Databases

In one embodiment, the identity and activity of the compounds are stored on a relational database. By evaluating the SAR data, lead compounds can be identified, and lead optimization libraries designed. The logically arranged arrays can be evaluated in a manner which automatically generates complete relational structural information such that a positive result provides: (1) information on a compound within any given spatial address on the multi-well plates and (2) the ability to extract relational structural information from negative results in the presence of positive results.

### II. Preparation of Recombinant Squalene Synthase

Squalene synthase can be produced in purified form by any known conventional techniques. For example, the DNA molecules encoding squalene synthase can be incorporated into cells using conventional recombinant DNA technology. The DNA molecules can be inserted into an expression system to which the DNA molecules are heterologous (i.e., not normally present) or where over-expression of the squalene synthase protein is desired.

For expression in heterologous systems, the heterologous DNA molecule is inserted into the expression system or vector in proper sense orientation and correct reading frame. The vector contains the necessary elements for the transcription and translation of the inserted protein-coding sequences. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

The nucleic acid sequences, or derivatives or truncated variants thereof can, for example, be introduced into viruses such as vaccinia virus. Methods for making a viral recombinant vector useful for expressing the squalene synthase protein are analogous to the methods disclosed in U.S. Pat. Nos. 4,603,112; 4,769,330; 5,174,993; 5,505,941; 5,338,683; 5,494,807; 4,722,848; Paoletti, E. (Proc. Natl. Acad. Sci. 93, 11349-11353 (1996)), Moss (Proc. Natl. Acad. Sci. 93, 11341-11348 (1996)), Roizman (Proc. Natl. Acad. Sci. 93, 11307-11302 (1996)), Frolov et al. (Proc. Natl. Acad. Sci. 93, 11371-11377 (1996)), Grunhaus et al. (Seminars in Virology 3, 237-252 (1993)) and U.S. Pat. Nos. 5,591,639; 5,589,466; and 5,580,859 relating to DNA expression vectors, inter alia; the contents of which are incorporated herein by reference in their entireties.

Recombinant molecules can be introduced into cells via transformation, particularly transduction, conjugation, mobilization, or electroporation. The DNA sequences are cloned into the vector using standard cloning procedures in the art, as described by Maniatis et al. (Molecular Cloning: A Laboratory Manual, Cold Springs Laboratory, Cold Springs Harbor, N.Y. (1982)), which is hereby incorporated by reference in its entirety.

A variety of host-vector systems can be used to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; vertebrate cell systems infected with virus (e.g., vaccinia virus, adenovirus, etc.); insect cell systems infected with virus (e.g., baculovirus) or fungal embryonic cells inoculated with the recombinant nucleic acid. The expression elements of these vectors vary in their strength and specificities. Depending upon the host-vector system used, any one of a number of suitable transcription and translation elements can be used.

Different genetic signals and processing events control many levels of gene expression (e.g., DNA transcription and messenger RNA (mRNA) translation). Transcription of DNA is dependent upon the presence of a promoter that is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals cannot be recognized in or cannot function in a prokaryotic system, and further, prokaryotic promoters are not recognized and do not function in eukaryotic cells.

Similarly, translation of MRNA in prokaryotes depends upon the presence of the proper prokaryotic signals that differ from those of eukaryotes. Efficient translation of mRNA in prokaryotes requires a ribosome binding site called the Shine-Dalgarno (SD) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer (Methods Enzymol. 68, 473 (1979)), which is hereby incorporated by reference in its entirety.

Promoters vary in their “strength” (i.e. their ability to promote transcription). For the purposes of expressing a cloned gene, it is desirable to use strong promoters to obtain a high level of transcription and hence, expression of the gene. Depending upon the host cell system used, any one of a number of suitable promoters can be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, can be used to direct high levels of transcription of adjacent DNA segments. Additionally, a hybrid trp-lacUV5 (tac) promotor or other E. coli promoters produced by recombinant DNA or other synthetic DNA techniques can be used to provide for transcription of the inserted gene.

Bacterial host cell strains and expression vectors can be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-β-D-galactoside). A variety of other operons, such as trp, pro, etc., are under different controls.

Once the isolated DNA molecule has been cloned into an expression system, it is ready to be incorporated into a host cell. Such incorporation can be carried out by the various forms of transformation noted above, depending upon the vector/host cell system. Suitable host cells include, but are not limited to, bacteria, virus, yeast, mammalian cells, and the like.

Recombinant expression vectors can be designed for the expression of the encoded proteins in prokaryotic or eukaryotic cells. The prokaryotic expression system can comprise the host bacterial species E. coli, B. subtilis or any other host cell known to one of skill in the art. Useful vectors can comprise constitutive or inducible promoters to direct expression of either fusion or non-fusion proteins. With fusion vectors, a number of amino acids are usually added to the expressed target gene sequence such as, but not limited to, a protein sequence for thioredoxin. A proteolytic cleavage site can further be introduced at a site between the target recombinant protein and the fusion sequence. Additionally, a region of amino acids such as a polymeric histidine region can be introduced to allow binding to the fusion protein by metallic ions such as nickel bonded to a solid support, and thereby allow purification of the fusion protein. Once the fusion protein has been purified, the cleavage site allows the target recombinant protein to be separated from the fusion sequence. Enzymes suitable for use in cleaving the proteolytic cleavage site include, but are not limited to, Factor Xa and thrombin. Fusion expression vectors that can be useful in the present invention include pGex (Arnrad Corp., Melbourne, Australia), pRIT5 (Pharmacia, Piscataway, N.J.) and pMAL (New England Biolabs, Beverly, Mass.), that fuse glutathione S-transferase, protein A, or maltose E binding protein, respectively, to the target recombinant protein.

Expression of unfused foreign genes in E. coil can be accomplished with recombinant vectors including, but not limited to, the E. coli expression vector pUR278 as described in Ruther et al. (E.M.B.O.J. 2, 1791-1794 (1983)), incorporated herein by reference in its entirety. Using the pUR278 vector, the nucleotide sequence coding for the pro1 gene product can be ligated in frame with the lacV coding region to produce a fusion protein.

Expression of a foreign gene can also be obtained using eukaryotic hosts such as mammalian, yeast or insect cells. Using eukaryotic vectors permits partial or complete post-translational modification such as, but not only, glycosylation and/or the formation of the relevant inter- or intra-chain disulfide bonds. Examples of vectors useful for expression in the yeast Saccharomyces cerevisiae include pYepSecl as in Baldari et al, (E.M.B.O.J. 6, 229-234 (1987)) and pYES2 (Invitrogen Corp., San Diego, Calif.), incorporated herein by reference in their entireties.

Baculovirus vectors are also available for the expression of proteins in cultured insect cells (F9 cells). Using recombinant Baculovirus vectors can be, or is, analogous to the methods disclosed in Richardson C. D. ed., (1995), “Baculovirus Expression Protocol” Humana Press Inc.; Smith et al (Mol. Cell. Biol. 3, 2156-2165 (1983)), Pennock et al., (Mol. Cell. Biol. 4, 399-406 (1984)) and incorporated herein by reference in their entireties.

III. Assay Methods

Methods for Quantifying Squalene

NADPH is consumed in a stoichiometric manner during squalene synthesis. The amount of squalene in a sample can be determined by following the decrease in concentration of NADPH.

The assay methods involve contacting FPP and NAPDH with squalene synthase and a magnesium ion cofactor. The reaction mixture is exposed to UV light, and the amount of NADPH over time is calculated based on the fluorescent light emitted by the NADPH. The amount of squalene is then calculated based on the amount of detected fluorescence.

Methods for Determining Squalene Synthase Activity

Squalene synthase activity can be determined in cell-free assays using isolated squalene synthase, preferably isolated recombinant squalene synthase, more preferably a water-soluble recombinant squalene synthase. Preferably, the squalene synthase is a truncated squalene synthase. The cell-free assays involve combining NADPH, FPP, squalene synthase and a magnesium ion cofactor to form a reaction mixture under conditions suitable for producing squalene, subjecting the reaction mixture to UV light and detecting fluorescent light emission. The amount of squalene produced can be determined by the amount of fluorescence, and the activity of the squalene synthase determined by the amount of squalene produced.

Squalene synthase activity can also be determined by in cell-based assays using the squalene synthase present in the cells, and the control amount of squalene produced by the cell (as measured by the loss in NADPH concentration) determined using a control. Cells can be lysed and the NADPH (and therefore squalene) measured in the lysate.

Methods for Identifying Herbicide/Fungicide/Insecticide Candidates

Test compounds suitable as herbicide, fungicide or insecticide candidates can be identified by combining NADPH, FPP, a magnesium ion cofactor and an appropriate squalene synthase from a plant or fungal source to form a reaction mixture in the presence and absence of the test compound. The effect of the compound on plants and fungi can be determined directly by the effect on squalene synthase. The effect of the compound on insects is determined indirectly by the effect on plant squalene synthase, because insects rely on plant sources of squalene to survive.

The reaction mixtures are subjected to UV light. The fluorescent light emission is detected and the amount of the fluorescent light emission in the presence and absence of the test compound is compared. An increase in the amount of fluorescent light emission over time in the presence of the test compound indicates that the test compound is a herbicide, fungicide or insecticide candidate.

In one embodiment, the compounds do not inhibit squalene synthase, but rather, promote squalene synthase. A decrease in the amount of fluorescent light emission over time in the presence of the test compound indicates that the test compound promotes squalene -synthase, and is therefore useful for plant or fungal growth.

Methods of Controlling Plant Growth

Chemicals, compounds or compositions identified by the above methods as modulators (i.e., promoters or inhibitors) of plant squalene synthase expression or activity can then be used to control plant growth. For example, compounds that inhibit plant growth can be applied to a plant or expressed in a plant to inhibit plant growth. Methods for inhibiting plant growth involve contacting a plant with a compound identified as having herbicidal activity.

Herbicides and herbicide candidates identified using the methods described herein can be used to control the growth of undesired plants, including both monocots and dicots. Examples of undesired plants include, but are not limited to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Compounds that promote squalene synthase activity can be used to promote plant growth. Such compounds can be desirable in the field of agriculture to increase crop yields.

Methods of Controlling Fungal Infection

Chemicals, compounds or compositions identified by the above methods as modulators of fungal squalene synthase expression or activity can then be used to control fungal infection. For example, compounds that inhibit fungal growth can be applied to an animal or plant or expressed in a plant, in order to prevent or treat fungal infections.

Accordingly, fungal infections can be treated or prevented by contacting a plant or animal with a compound identified by the methods of the invention as having fungicidal activity.

Methods of Selectively Inhibiting Squalene Synthase

Methods for identifying compounds that can selectively inhibit squalene synthase activity are particularly useful. Compounds that selectivity inhibit plant, fungal or animal squalene synthase activity, in preference to other squalene synthase activity, can be used to identify compounds useful to target fungi and/or animals over plants, plants over fungi and/or animals, or bacteria over fungi and/or animals.

A suitable squalene synthase for use in the assays is derived from Arabidopsis thaliana, wherein the squalene synthase has the amino acid shown in sequence SEQ ID NO: 5, or a derivative or truncated version thereof.

In one embodiment, potential herbicidal compounds are evaluated with respect to their ability to inhibit squalene synthase in animals or fungi that adversely affect plants. Ideally, the compounds either do not adversely affect the squalene synthase in the plants of interest, or do so to a lesser degree. This can be determined, for example, by preparing or obtaining an appropriate library of compounds, screening them for activity against a suitable plant squalene synthase, and then screening them for activity against a suitable fungal or animal squalene synthase. Compounds that are selective for the fungus or animal over the plant of interest can then be identified.

Methods of Inhibiting the Formation of Squalene Synthase

The total amount of squalene produced by an animal, plant or fungus can be altered by affecting the formation of squalene synthase itself or by modulating squalene synthase activity after the squalene synthase is formed. Cell free assays use the squalene synthase and focus on compounds that inhibit the activity of the squalene synthase. Cell based assays can be used to identify compounds that effect squalene synthase formation as well as compounds that effect the squalene synthase once formed. Compounds identified in the cell based assays can be used to alter squalene synthase formation, or alter the squalene synthase that is formed. Because enzyme production is controlled by DNA, nucleic acids are one example of compounds that can be used to alter squalene synthase expression.

Accordingly, isolated “antisense” nucleic acids can be used as “antisense” fungicides and/or herbicides. An antisense construct can be delivered, for example, as an expression plasmid that when transcribed in the fungal or plant cell, produces RNA that is complementary to at least a unique portion of the cellular mRNA which encodes a squalene synthase protein. Alternatively, the antisense construct can be an oligonucleotide probe that is generated ex vivo and, when introduced into the fungal cell, inhibits expression by hybridizing with the mRNA and/or genomic sequences encoding one of the subject squalene synthase proteins.

Uses for Squalene Synthase Inhibitors

Squalene synthase inhibitors discovered using the assay methods described herein can be used, for example, as herbicides when they inhibit plant squalene synthase, fungicides when they inhibit fungal squalene synthase, and to lower cholesterol and mediate steroid-related bioactivities when they inhibit mammalian, particularly human, squalene synthase. Squalene synthase inhibitors have also been suggested for use in treating Alzheimer's disease, inhibiting bone resorption, inhibiting hair growth, inhibiting acne, preventing embryonic growth retardation and neural tube defects, and in treating and/or preventing tumors, particularly cancerous tumors.

IV. High Throughout Methodology

The assays used to measure squalene synthase activity can be generated in many different forms and include assays based on cell-free systems, e.g. purified proteins or cell lysates, as well as cell-based assays that use intact cells. In order to test libraries of compounds and natural extracts, high throughput assays are desirable to maximize the number of compositions surveyed in a given period of time.

Assays performed in cell-free systems, such as can be derived with purified or semi-purified proteins or polypeptides thereof or with lysates, are often preferred as “primary” screens in that they can be generated to permit rapid development and relatively easy detection of an alteration in a molecular target which is mediated by a test composition. The effects of cellular toxicity and/or bioavailability of the test composition can be generally ignored in the in vitro system, the assay instead being focused primarily on the effect of the drug on the molecular target as can be manifested in an alteration of binding affinity with other proteins or change in enzymatic properties of the molecular target.

Potential inhibitors of the enzyme activity can be detected in a cell-free assay generated with an isolated squalene synthase enzyme in a cell lysate or an isolated squalene synthase enzyme purified from the lysate. Some of the compounds will bind directly to the target polypeptide, and these can be identified using competitive and non-competitive binding assays, Scatchard plot determinations, and the like.

Microarrays can be used to test a large number of compounds using a minimum amount of laboratory space. The term “microarray” as used herein refers to an arrangement of distinct polynucleotides or peptides or polypeptides arranged on a substrate, e.g. paper, nylon, any other type of membrane, filter, chip, glass slide, silicone wafer, or any other suitable solid or flexible support.

Multiwell plates, for example, 96- and 384-well plates, can be used to run multiple assays at the same time. Liquid handlers, for example, those sold by Tecan, can be used to add repeatable amounts of small volumes of liquid to each of the wells. High throughput analytical equipment can be used to analyze multiple samples in a relatively short amount of time. Relational databases, as such are known in the art, can be used to store information about the structure and activity of the compounds that are analyzed.

The conditions for one embodiment of the high-throughput bioassays described herein are as follows: A fluorometric high throughput assay for detecting squalene synthase inhibitory activity was developed in 384-well microtiter plate format. Recombinant squalene synthase from E coli sources are suitable and can be used in the assays.

The substrates (NADPH, magnesium ion cofactor and FPP) are mixed in a buffer solution (i.e., phosphate buffer, pH 7.5) containing 50-1,000 ng of recombinant protein in a total volume of about 50 μl.

The bioassays are preferably performed using robotic systems such as are commonly used in combinatorial chemistry. Enzyme inhibition can be measured via fluorescent detection. Fluorescence readings can be taken at an excitation wavelength of 340 nm and an emission wavelength of 465 nm, for example, on a Tecan Ultra reader (Tecan), which supports all plate types (from 6 well up to 1536 well), has a relatively short measurement time for all plate formats: <1 min (uHTS), and has a wavelength range from 230 nm to 850 nm.

Combinations of stock solutions at standard concentration can be prepared for the automated steps of the synthesis. The compounds to be evaluated can be solubilized in any suitable solvent, for example, dimethyl sulfoxide (DMSO) and pre-transferred to a multi-well plate (for example, a 96 or 384 well assay plate) to yield the indicated final concentration of compound.

The number and percentage (i.e., “hit rate”) of compounds in each array that produce greater than 50% inhibition can be determined for each array.

The percentage of inhibition can be plotted against the logarithm of inhibitor concentration, and the inhibitor concentration at 50% inhibition can be determined (IC₅₀).

The discovery of potential herbicides, insecticides and/or fungicides can be accelerated by integrating high throughput testing with high throughput synthesis and/or by using logically ordered, spatially addressable arrays.

Methods of Preparing and Arranging Combinatorial Libraries

Combinatorial libraries of compounds to be evaluated using the bioassays described herein can be prepared using known methods, for example, by reacting components to form a molecular core structure and structural diversity elements. Thus, during synthesis, “components” are used to make the “members” or “individual compounds” of an array, and the terms “molecular core” (or “molecular core structure”) and “diversity element” (or “structural diversity element”) are used herein to describe the parts of the completed compounds of an array.

The members of the new arrays can be constructed from a wide variety of reaction components. Each component can form a part or all of a molecular core structure or structural diversity element. Thus, components can be added to reactive sites on a preexisting molecular core structure to form or attach structural diversity elements.

On the other hand, the molecular core structure and the structural diversity elements can, in some cases, be formed from a combination of two or more components. For example, one component can include a portion of a molecular core structure and also a partial or complete structural diversity element, while a second component can include the remainder of the molecular core structure together with any remaining structural diversity elements.

The methods described above can also be used to synthesize libraries of compounds to be used in the construction of an array. Laboratory-scale robotic devices can be used to automate the unit operations of the organic chemical syntheses. The analysis of the synthesis products can be integrated into automated synthesis as an on-line quality control function, with automated data acquisition and storage, and historical process analysis.

A 96-well or 384-well microtiter-type spatial format plate can serve as the foundation for managing both high throughput screening data and chemical synthesis data. Organic compounds arrayed in alpha-numerically registered 96-well or 384-well plates can be specified by descriptors derived from row, column, and plate numbers. The descriptors are ideally suited for electronic storage and retrieval from chemical and biological databases. This format allows high throughput bioassays for inhibiting or promoting squalene synthase to be performed with the chemical arrays and provides insights into structure activity relationships of the chemical arrays.

The present invention will be better understood with reference to the following non-limiting examples.

### EXAMPLE 1

Generation of Recombinant Squalene Synthase

Antisense technology can be used to suppress squalene synthase activity in Arabidopsis. The suppressed squalene synthase activity shows that squalene synthase activity is essential for Arabidopsis growth and development.

This experiment illustrates the generation of suitable recombinant squalene synthase proteins for use in the assays described herein. It should be noted that other squalene synthase proteins than those specifically described herein can be used in the assays.

Cloning strategy -C-terminal truncated Squalene Synthase (tSQS) (removal of 22 a.a. from C-terminal):

Total RNA was collected from 14 day old Arabidopsis thaliana seedlings using published protocols and reagents (TRIZOL™) from Life Technologies, Inc. (Rockville, Md.). 1 microgram of total RNA was incubated with 10 pmol of custom oligo, 5′-GGA ATT CTC ATG GTT GTC. CTT TGT CAT TAA C-3′ (SEQ ID NO: 7), in a reverse transcription reaction (ThermoScript RT kit, Life Technologies, Inc.) according to the manufacturer's recommendations. Polymerase chain reaction (PCR) was carried out in a total volume of 50 μl with the following reagents: two μl of above RT reaction mixture, 20 mM Tris-HCl pH 8.8, 2 mM MgSO₄, 10 mM KCl, 10 mM (NH₄)₂SO₄, 0.1% Triton X-100, 0.1 mg/ml BSA, 0.8mM deoxyribonucleotide triphosphates, 50 pmol of each primer (5′-CGG GAT CCA TGG GGA GCT TGG GGA CGA T-3′ (SEQ ID NO: 8) and 5′-GGA ATT CTC. ATG GTT GTC. CTT TGT CAT TAA C-3′ (SEQ ID NO: 9)) and 2.5 units Pfu polymerase (Stratagene, USA).

PCR cycling was as follows: 94° C. (30 sec), 60° C. (1 min), 72° C. (2 min) with each cycle decreasing annealing temperature at 0.5° C., 10 cycles of touch down PCR starting at 94° C. (30 sec), 50° C. (1 min), 72° C. (2 min) with each cycle decreasing annealing temperature at 0.5° C. The resulting PCR product and plasmid pET30a(+) (Novagen, Madison, Wis.), were digested with restriction endonucleases BamH l and EcoRl, as directed by the manufacturer (Life Technologies, Inc.). The PCR product is shown in SEQ ID NO: 5, representing the nucleotide sequence of the truncated SQS gene used for this study. The translation of this nucleotide yields the squalene synthase identified in SEQ ID NO: 6.

The DNA encoding the N-terninal peptide fusion, provided by the pET30a(+) vector, that encode a 6HIS tag, thrombin cleavage site, S-tag, and enterokinase site, in that order, are a 150 base pair oligonucleotide (SEQ ID NO: 10, which encodes a 50 amino acid protein (SEQ ID NO: 11).

Ligation of these two linear DNAs into the resulting recombinant clone pET30/tSqs (1317 nucleotides, SEQ ID NO: 12) was accomplished by following instructions included with T4 DNA ligase (Life Technologies, Inc.). Integrity of the above clone was verified by DNA sequence analysis. The translation of sequence ID No. 12 yields fusion protein pET30/tSqS (SEQ ID NO: 13).

Methods used to express the squalene synthase gene:

Clone pET30/tSqs was transformed into a proprietary bacterial strain, E. coli BL21(DE3)lysS (Novagen, Madison, Wis.), following the manufacture's instructions. Transformed bacteria were grown in LB liquid media (10 grams each tryptone and NaCl; 5 grams yeast extract; H₂O to one liter) supplemented with 34 micrograms/milliliter chloramphenicol and 50 micrograms/milliliter kanamycin, at 37° C. to an optical density of 0.6 at 600 nm. At that point, isopropylthio-Beta-galactoside was added to a final concentration of 1 millimolar and the culture was incubated at 23° C. for 16 additional hours. Bacteria were pelleted via centrifugation, the supernatant discarded, and the pellet frozen to −80 C. Pellets were resuspended in 50 mM Tris pH 7.5, 20 mM MgCl2, 0.3M NaCl, 1 m DTT, and EDTA-free Protease Inhibitor Cocktail (Boehringer-Mannheim, as directed). Collected supernatant contained soluble squalene synthase protein, as determined by western blot analysis. The protein includes 388 amino acids, and the sequence is provided as SEQ ID NO: 6.

Expression and Purification of SQS

Optimization of SQS Expression and Purification:

Four different E.coli expression constructs were analyzed for expression of SQS, 1) A full length sequence which included an N-terminal HIS tag, 2 and 3) A C-terminal truncated sequence which removed a putative membrane anchor region. This truncated sequence (SEQ ID NO: 5) was used to form both N-terminal and C-terminal HIS tagged constructs, 4) A full length sequence, which contained no affinity tag. Experiments showed that the N-terminal HIS-tagged truncated version of the gene gave the highest levels of soluble protein expression and that the enzyme was highly active.

The truncated SQS sequence containing an N-terminal HIS tag was expressed in E.coli, and purified by Ni-chromatography. The resulting protein sample was tested with an Agilent 2100 Bioanalyzer. A sample from the elution showed a major peak comprising ˜80% of the total protein in the sample. All further work with SQS was done with this expression vector.

Expression and Purification of SQS: E.coli cultures were grown at 37° C. to an optical density of ˜0.6. IPTG was added to a final concentration of 1 mM to induce recombinant protein expression, and the culture allowed to continue for an additional 16 hours at room temperature. Cells were then harvested by centrifugation at 7,000 rpm for 10 minutes. The 30-liter fermentation was first concentrated down to ˜5 liters with a 0.22 μm hollow fiber tangental flow filter. After centrifugation, the cells were stored at −80° C. Cell pellets were resuspended in Bugbuster+benzonase (Novagen, Madison, Wis.) to lyse the cells, followed by centrifugation at 15,000×g for 10 minutes to clarify the lysate. Clarified lysate was then applied to a nickel column (Qiagen). The column was washed with 3 column volumes of buffer (50 mM phosphate buffer, pH 7.5, 500 mM NaCl) containing 20 mM imidazole, followed by an additional 3 column volume wash containing 50 mM imidazol. Recombinant protein was then eluted with 500 mM imidazol. Protein fractions were pooled, and the resulting solution was desalted by gel filtration. Final protein concentration was determined (BioRad) and the solution frozen and stored at −80° C. The optimal protein concentration per assay well was determined by titration to be 100- 125 ng/well. Enzyme activity proved to be stable up to at least 25 days of storage.

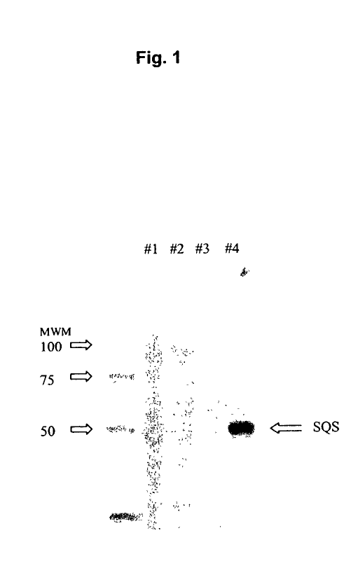

Samples were resolved by SDS-PAGE, then transferred to nitrocellulose. Blots were probed for the 6XHIS affinity tag with a mouse anti-penta-HIS antibody (Qiagen), followed by detection with a rabbit anti-mouse alk.phos. conjugated secondary. Visualization was with NBT/BICP. The results are shown in FIG. 1, where lane 1 represents the whole cell lysate, lane 2 the clarified lysate, lane 3 the column flow through, and lane 4 the elution. The column and storage buffer were 50 mM sodium phosphate buffer pH 7.5 and 500 mM NaCl.

### EXAMPLE 2

Validation of Activity by Fluorescence Assay (340 ex/465 em)

In literature to date, the methods used to determine the activity of squalene synthase involved radiolabelled FPP and a direct measurement of degradations per minute. Here, a fluorometric assay was created which indirectly measures squalene synthesis by measuring the amount of NADPH used in the conversion of FPP to squalene.

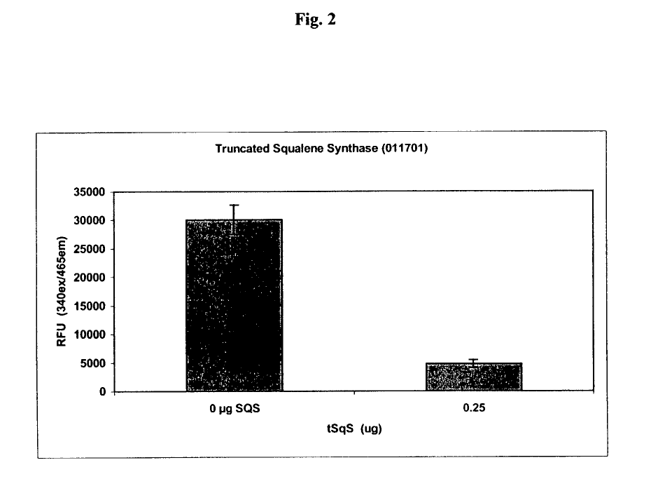

In this example, 25 μl of diluted, purified SqS (250 ng) was incubated for 1 hour at 37° C. with 25 μl substrate (FPP)/NADPH (final concentrations 60 μM and 25 μM respectively). The results (shown in FIG. 2), demonstrate suggest that the recombinant SqS was active in converting FPP to squalene.

The purified enzyme was obtained from a 50 ml E. coli culture expressing C-terminal truncated N-His tagged squalene synthase from a pET30a vector. The total assay volume was 50 μl containing 60 μM FPP, 25 μM NADPH, 20 mM MgCl₂, 1 mM DTT, 1 mg/ml BSA and 50 mM Tris pH 7.5. The values shown in FIG. 2 are the mean of triplicate determinations, with the standard error shown as error bars. EXAMPLE 3

Validation of Activity by GC-MS

Nickel/NTA column-purified SqS enzyme was isolated from a 1 liter cell culture. 25 μl of enzyme extract (2 μg) were incubated with 25 μl of 500 μM substrate and 500 μm NADPH for 1 hours at 37° C. The SqS reaction was analyzed by the Tempus GC-MS using squalene and FPP as standards. The physical properties of squalene prohibited analysis via HPLC. In the presence of active enzyme, FPP was converted to squalene.

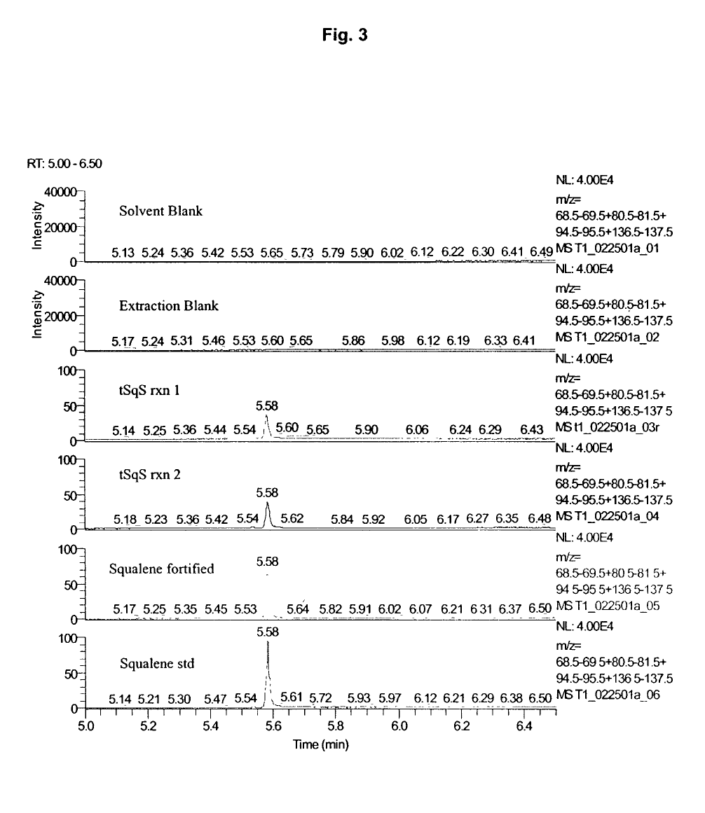

The results are shown in FIGS. 3A-3F, which are representative GC-MS spectra. Solvent and extraction blanks (FIGS. 3A and 3B, respectively), did not show any squalene. FIGS. 3C. and 3D represent the results of two reactions, where 50 μl of 50:50 MTBE/hexane was added to 250 μl of the tSqS reaction sample. The sample was vortexed and 25 μl of the organic layer extracted and injected into the instrument. Control squalene injection showed all squalene was recovered (FIG. 3E). Results are compared to a 10 μg squalene standard (FIG. 3F).

### EXAMPLE 4

Optimization of HT Parameters-Squalene Synthase Titration

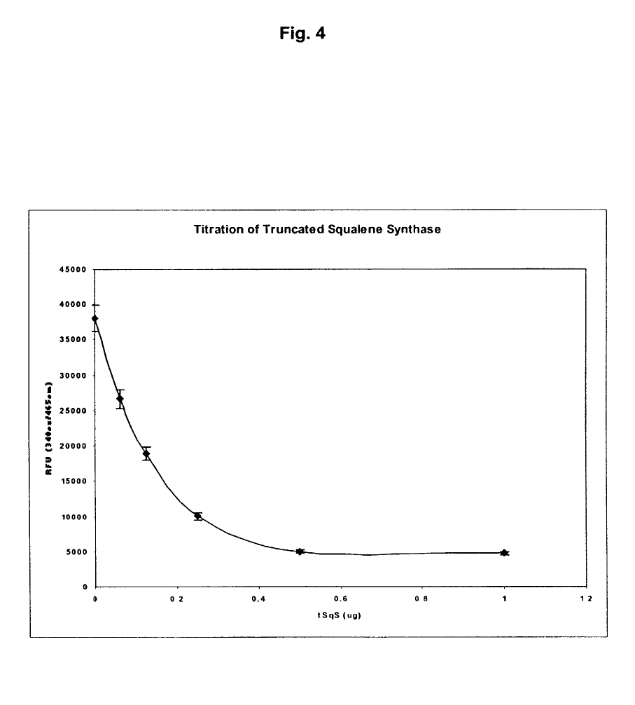

Purified tSqS was diluted with 50 mM Tris pH 7.5, 5 mM MgCl₂ and 1 mg/ml BSA, 1 mM DTT to different concentrations and the squalene synthase activities were determined. The assays were performed using 40 μM substrate, and the results are shown in FIG. 4. Substrate and NADPH (40 and 10 μM final concentrations, respectively) were incubated with purified enzyme or assay buffer for 30 min at 37° C. Total assay volume was 50 μl. Instrument gain was set on the no enzyme control at time of enzyme addition. Values are the mean of triplicate determinations, with standard error shown as error bars.

The data show that as the tSqS concentration increased, the fluorescence (RFU) decreased. The decreased fluorescence represents a decrease in NADPH concentration and a correlating increase in squalene concentration. A good linear relationship existed to 0.15 μg protein per assay well using 40 μM FPP. The signal to noise ratio was approximately 2-fold at this amount. Adjustment of the magnesium concentration further increased this window.

### EXAMPLE 5

Time Course of Squalene Synthase at 40 μM FPP

The experiment was performed to optimize the concentration of the tSqS enzyme. Various amounts of purified SqS were incubated over time at 37° C. with 40 μM FPP and 10 μM NADPH in assay buffer containing 50 mM Tris pH 7.5, 5 mM MgCl₂, 1 mg/ml BSA, and 1 mM DTT. Instrument gain was set on the no enzyme control at time of enzyme addition. Values are the mean of triplicate determinations.

The results show that the reaction was complete in about 20 minutes when the about 1 μg tSqS was used, about 120 minutes when about 0.06 μg tSqS was used, and intermediate times when intermediate amounts were used. Based on this information, 100 ng tSqS (30 minute reaction times) was used for further experiments, and 80 ng tSqS (30 minute reaction times) was used for screening.

### EXAMPLE 6

Km determination for FPP.

This experiment was performed to determine the Km for FPP. The Km for FPP was determined by varying the FPP concentration at saturating NADPH. Readings were taken every 2 minutes while incubating for 1 hour at 37° C. Because the reaction catalyzed by squalene synthase adds both substrate molecules sequentially, and being an NADPH depletion assay, traditional Michaelis-Menten kinetics do not give an accurate measurement of Km.

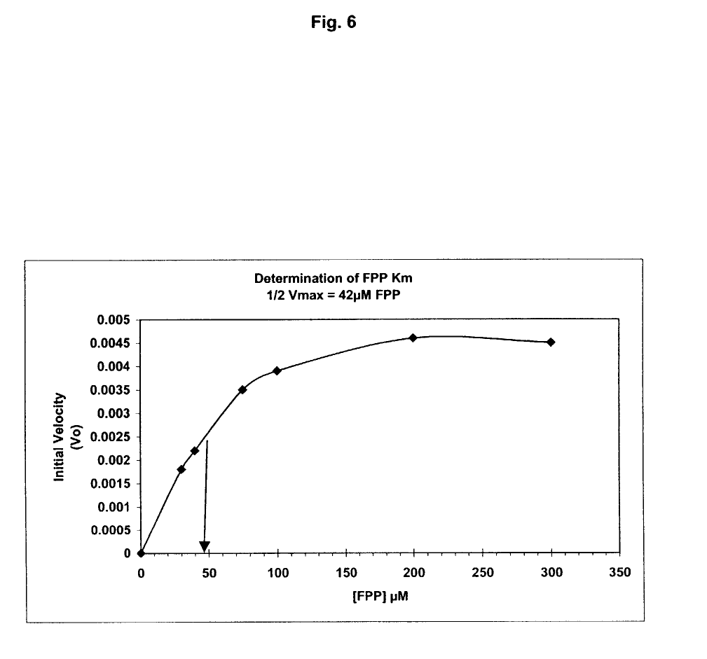

For this assay, the initial velocity for each FPP concentration was determined and plotted in FIG. 6. The Km was calculated to be about 42 μM. This value differs slightly with Km values found in the literature referring to yeast, E. coli, rat and human liver microsomes (LoGrasso et al., Arch. Biochem. Biophys. 307(l):193-199 (1993), Zhang et al., Arch. Biochem. Biophys. 304(1):133-143 (1993), Kuswik-Rabiega et al., J. BioL. Chem., 262(4):1505-1509 (1987), Soltis et al., Arch. Biochem. Biophys. 316(2):713-723 (1995), Kroon et al., Phytochemistry, 45(6):1157-1163 (1997) and Nakashima et al., Proc. Natl. Acad. Sci., 92:2328-2332 (1995)). However, the similarity in these SqS sequences has been described as “poor” (Kribii et al., Eur. J Biochem., 249(l):61-69 (1997)), and it is believed there has been no Arabidopsis Km_{FPP} data reported in literature to date.

FPP concentrations were varied as indicated in FIG. 6. Assays were performed in a 50 μl total volume with 50 ng tSqS, 100 μM NADPH, 10 mM MgCl₂, 1 mM DTT, 1 mg/ml BSA and 50 mM Tris/HCl pH 7.5. Reactions were run for 1 hour at 37° C. Values are the mean of 3 determinations.

### EXAMPLE 7

Influence of NADPH on the Assay

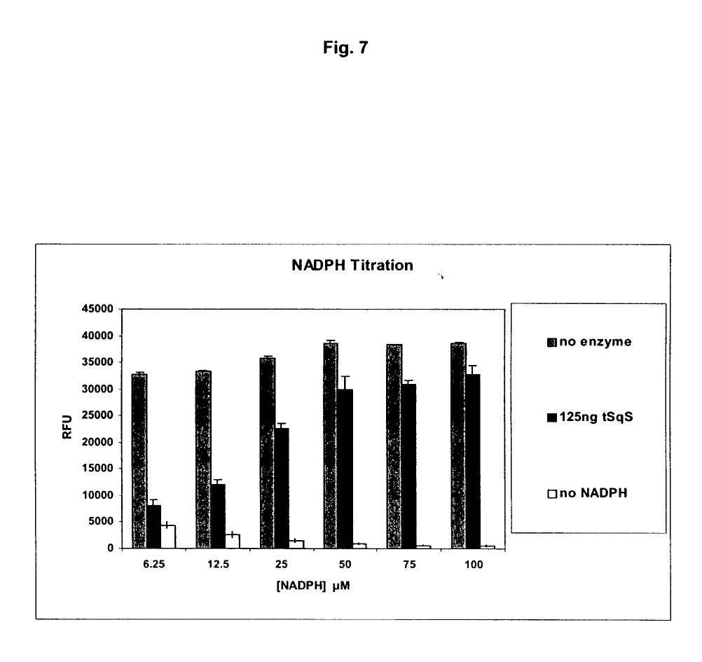

This experiment was performed to determine the optimum NADPH concentration for use in the high throughput assays. The effect of NADPH upon the signal:noise ratio of the TECAN Ultra was measured using its gain (as shown in FIG. 7). At concentrations below 6 μM NADPH, the instrument automatically increases the gain, thus raising the background and error in the assay. At much higher levels, too much NADPH is present for the TECAN Ultra to detect the changes in NADPH depletion. Here, a careful balance must be reached for this type of assay and instrument. This experiment was repeated twice at a broader and more narrow range, with the graph in FIG. 7 being the most representative of all the data collected.

Various concentrations of NADPH were incubated for 30 minutes at 37° C. in a 50 μl assay. Final concentrations were 40 μM FPP, 125 ng SqS, 10 mM MgCl₂, 1 mM DTT, 1 mg/ml BSA and 50 mM Tris/HCl pH 7.5. The instrument gain was set at the time of enzyme addition on the no enzyme control for each concentration of NADPH. Values are the mean of triplicate determinations, standard deviation is indicated by error bars.

The data show that for maximal activity in this assay, 10 μM NADPH is recommended. This is in contrast to the theoretical reaction stoichiometry of 2 moles FPP to 1 mole NADPH. However, it should be noted that when different instrumentations is used, the optimum value would be expected to vary. Those of skill in the art, taking into consideration the teachings provided herein, can readily determine an optimum NADPH concentration for use in the high throughput assays described herein using a particular analytical device.

### EXAMPLE 8

Titration of Magnesium Cofactor

The second step in the enzymatic formation of squalene involves using NADPH and the Mg⁺² cofactor to reduce the intermediate PSPP. This experiment was performed to determine optimum magnesium ion concentrations for use in the high throughput assay.

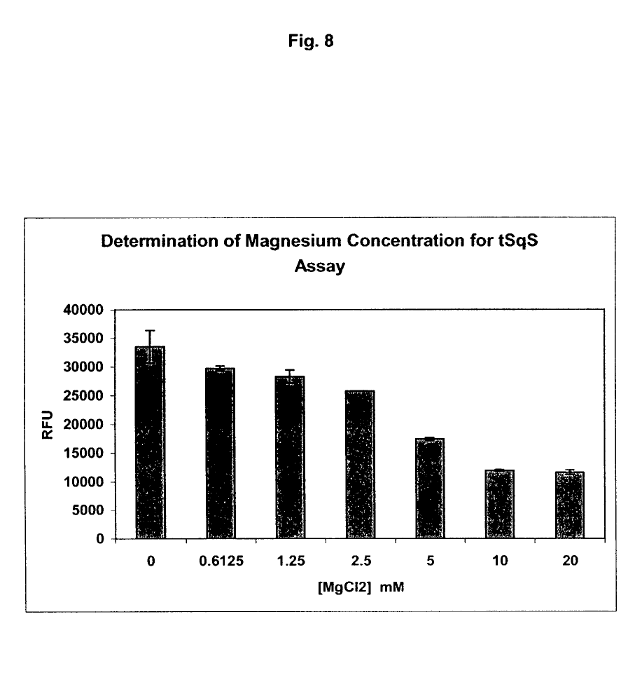

SqS activity was measured with a 50 μl total volume reaction containing 10 μM NADPH, 40 μM FPP, 50 mM Tris pH 7.5, 1 mg/ml BSA, and 1 mM DTT using decreasing amounts of MgCl₂ in the reaction mixture. The reaction mixture was incubated for 30 minutes at 37° C. The data are shown in FIG. 8.

The data show that low magnesium ion concentrations (less than about 5 mM) are most likely insufficient to complete the reaction. High concentrations of magnesium ions (above about 20 mM) begin to interfere with the assay as well as precipitate the substrate (data not shown). A final concentration of 10 mM was chosen for high throughput synthesis development, where the signal:noise ratio was greatest. The optimum concentration would be expected to vary if different instrumentation or concentrations of other components were used. Those of skill in the art, taking into consideration the teachings provided herein, can readily determine an optimum magnesium ion concentration for use in the high throughput assays described herein using a particular analytical device or using different reactant concentrations.

### EXAMPLE 9

Effect of Temperature on SqS Activity

This experiment was performed to determine the optimum incubation temperature for performing the assay. Incubations at room temperature (RT) and 37° C. were compared. The experiment was performed in a total volume of 50 μl with 125 ng SqS, 40 μM FPP, 10 μM NADPH, 50 mM Tris/HCl pH 7.5, 10 mM MgCl₂, 1 mM DTT, 1 mg/ml BSA. Reaction mixtures were incubated for 30 minutes at either room temperature or 37° C.

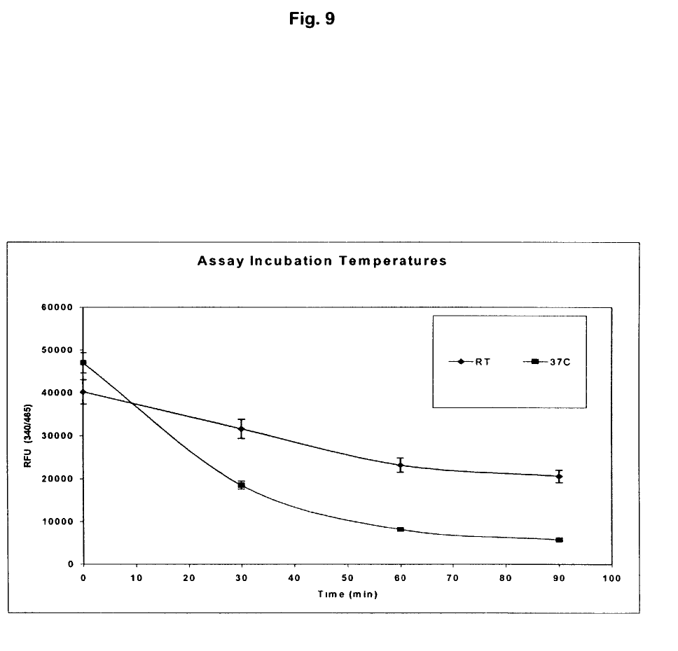

The effect of incubation temperature on SqS activity is shown in FIG. 9. Values are the mean of triplicate determinations, with standard error indicated by error bars. The data indicate that the optimal reaction temperature is 37° C.

### EXAMPLE 10

Effect of Temperature on Assay Reagents.

This experiment was performed to observe the effect of varying the temperature of the assay reagents (FPP, NADPH and tSqS) before combining them in the reaction mixture. 80 μM FPP, 20 μM NADPH, and 0.005 μg/μl tSqS were pre-incubated for 5 minutes at 4° C., room temperature and 37° C. before combining the reagents into the 50 μl assay.

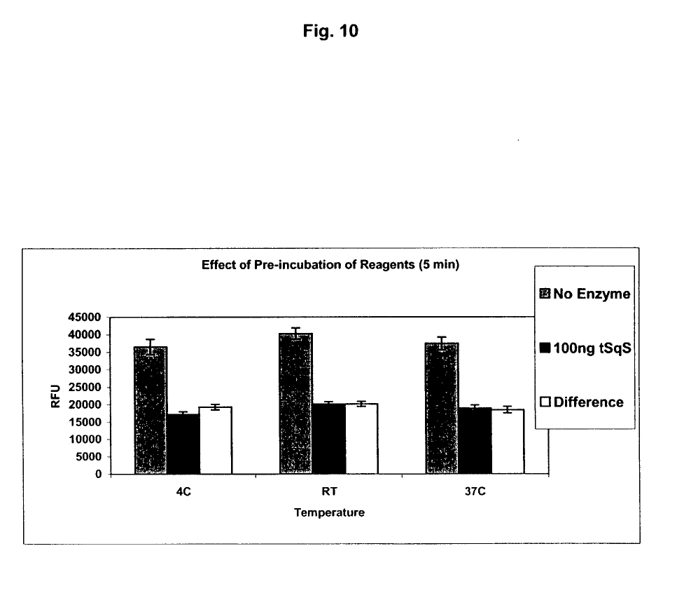

The results are shown in FIG. 10. Virtually no difference was observed when the reagents were pre-incubated at temperatures in the range of 4 to 37° C. EXAMPLE 11

Incubation of SqS and Substrate over Time.

This experiment was performed to observe the decrease in fluorescence over time when the reaction mixture included tSqS and did not include tSqS, to determine whether the fluorescence due to NADPH would decrease over time in the absence of squalene synthase.

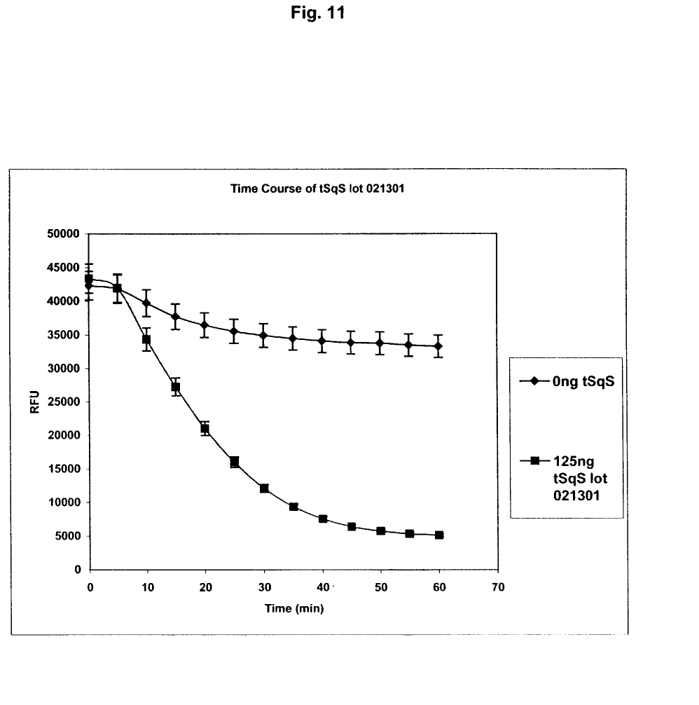

10 μM NADPH was incubated with 40 μM FPP over time at 37° C. in the presence and absence of 125 ng purified enzyme. The instrument gain was set on the no enzyme control. The data is shown in FIG. 11, where the values are the mean of triplicate determinations and standard error is shown as error bars. The data show that there is a slight decrease in fluorescence over time when the NADPH is kept at 37° C.

### EXAMPLE 12

Lot-to-Lot Comparison of tSqS

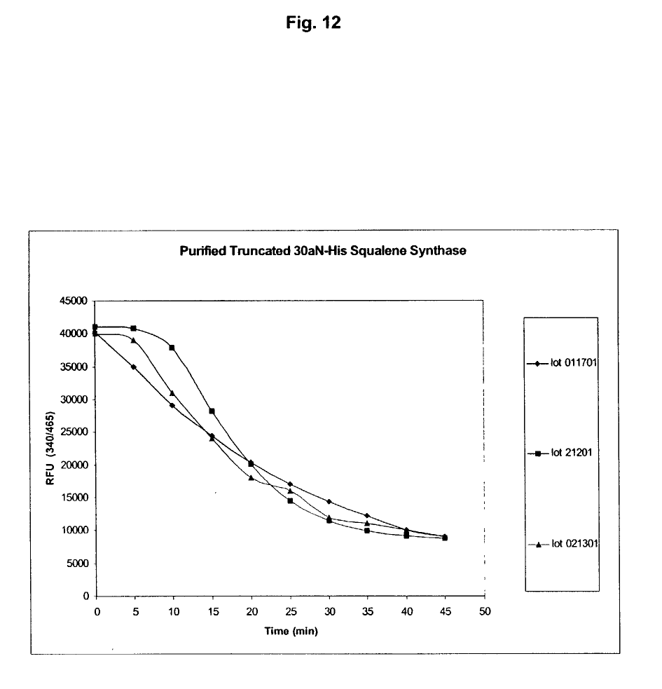

This experiment was performed to evaluate three separate lots of tSqS prepared as described in EXAMPLE 1. 100 ng of tSqS was incubated with 40 μM FPP, 10 μM NADPH, 10 mM Mg, 1 mg/ml BSA and 1 mM DTT in 50 mM Tris pH 7.5 at 37° C. and the fluorescence (RFU) was monitored over time (0 to 45 minutes). The data is shown in FIG. 12. The initial rates of reaction varied slightly. However, at the 30 minute time point the lots were nearly equal in activity.

### EXAMPLE 13

BSA Effects

The effect of bovine serum albumin (BSA) on the assay was evaluated to improve the linear relationship with the enzyme amount as well as the Z-factor. 40 μM FPP 10 μM NADPH were incubated with varying BSA concentrations (between 0 and 1 mg/ml) in assay buffer for 30 minutes at 37° C. in the presence and absence of 125 ng tSqS. The total assay volume was 50 μl.

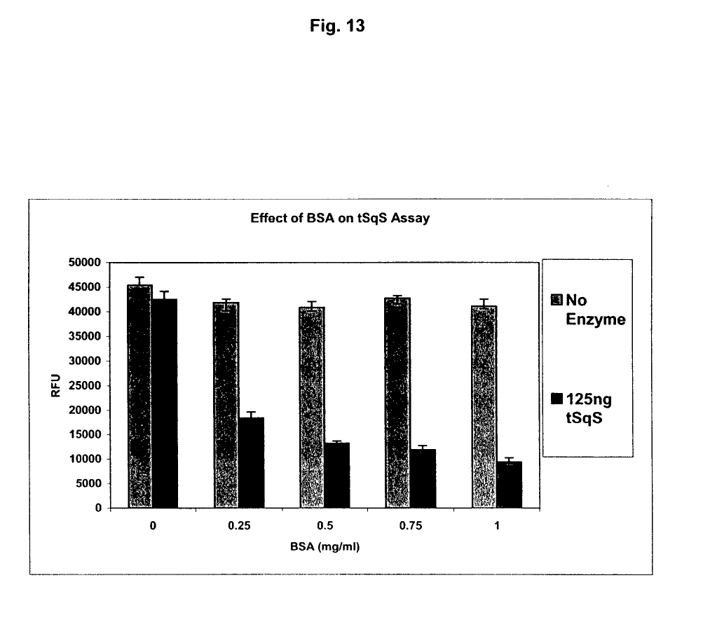

The results are shown in FIG. 13, where the values are the mean of triplicate determinations, with standard error shown as error bars. The data show that it is preferable to add at least 0.25 mg/ml BSA for optimum assay activity. The effect is most likely due to the low amounts of total protein present.

### EXAMPLE 14

Substrate Stability at 4° C.

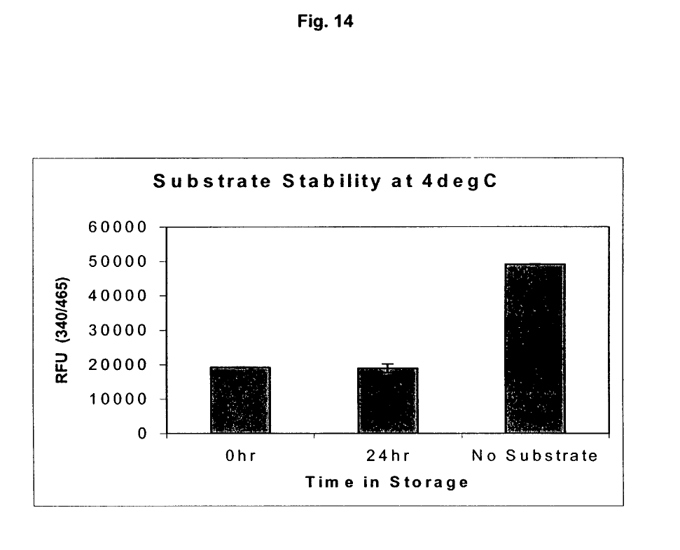

This experiment was performed to evaluate the stability of the FPP when stored for 24 hours at 4° C. The enzymatic activities using freshly made substrate and 24 hour stored substrate were compared. 80 μM FPP was stored at 4° C. for 24 hours. The reaction mixture included 40 μM substrate, 10 μM NADPH, 125 ng enzyme and assay buffer in total volume of 50 μl. The incubation time was 30 minutes at 37° C. The data is shown in FIG. 14, where the values are the mean of triplicate determinations, with standard error shown. The data show that no significant loss of activity was observed, indicating that FPP was stable at 4° C. for 24 hours.

### EXAMPLE 15

Substrate Stability at Room Temperature

This experiment was performed to evaluate the stability of the FPP when stored for 24 hours at room temperature. The enzymatic activities using freshly made substrate and 24 hour stored substrate were compared.

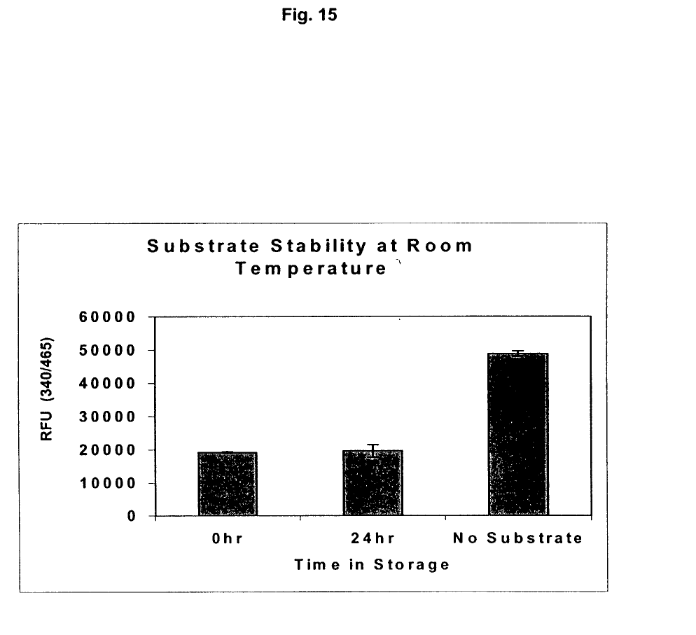

80 μM FPP was stored at room temperature for 24 hours. The reaction mixture included 40 μM substrate, 10 μM NADPH, 125 ng enzyme and assay buffer in total volume of 50 μl. The incubation time was 30 minutes at 37° C. The data is shown in FIG. 15, where the values are the mean of triplicate determinations, with standard error shown. The data show that no significant loss of activity was observed, indicating that FPP was stable at room temperature for 24 hours.

### EXAMPLE 16

Enzyme Stability at 4° C.

This experiment was performed to evaluate the stability of the tSqS when stored for 24 hours at 4° C. The enzymatic activities using freshly made enzyme and 24 hour stored enzyme were compared.

0.005 μg/μl tSqS was stored at 4° C. for 24 hours and then used directly in the assay in a comparison with freshly prepared tSqS. The reaction mixture included 40 μM substrate, 10 μM NADPH, 125 ng enzyme (tSqS) and assay buffer in a total volume of 50 μl. The incubation time was 30 minutes at 37° C.

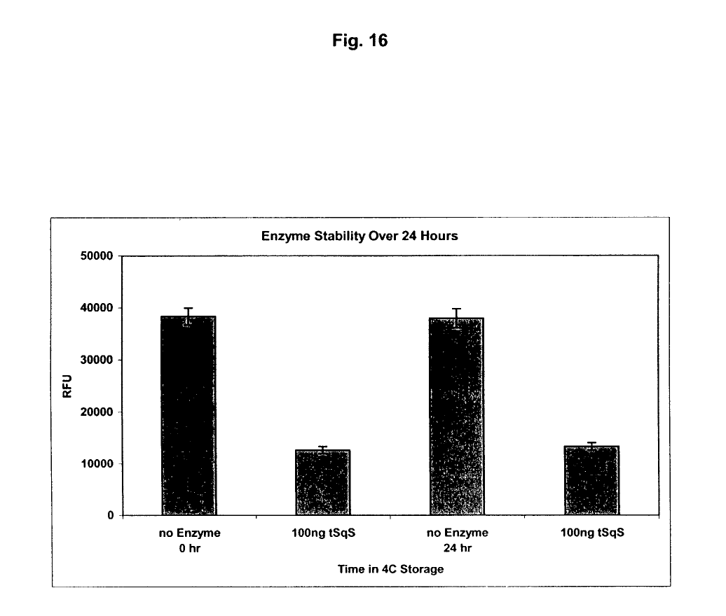

The data is shown in FIG. 16, where the values are the mean of triplicate determinations with standard error shown. No significant activity loss was observed, indicating that the enzyme is stable at 4° C. over a period of 24 hours.

### EXAMPLE 17

Enzyme Stability at Room Temperature

This experiment was performed to evaluate the stability of the tSqS when stored for 24 hours at room temperature. The enzymatic activities using freshly made enzyme and 24 hour stored enzyme were compared.

0.005 μg/μl tSqS was stored at room temperature for 24 hours and then used directly in the assay in a comparison with freshly prepared tSqS. The reaction mixture included 40 μM substrate, 10 μM NADPH, 125 ng enzyme (tSqS) and assay buffer in a total volume of 50 μl. The incubation time was 30 minutes at 37° C.

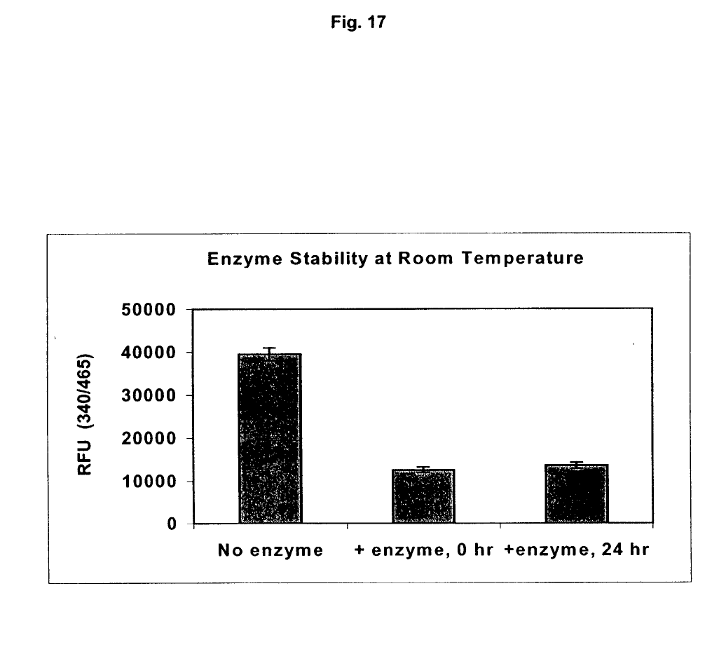

The data is shown in FIG. 17, where the values are the mean of triplicate determinations with standard error shown. No significant activity loss was observed, indicating that the enzyme is stable at room temperature over a period of 24 hours. However, as for most enzymes, it is recommended that during the assay run, SqS be stored at 4° C. until use.

### EXAMPLE 18

Enzyme Stability Under Various Conditions.

This experiment was performed to evaluate the effect of storage conditions and freeze/thaw cycles on the tSqS. 100 μl enzyme was aliquoted and stored at various conditions to determine loss of activity.

The conditions included a) one freeze/thaw cycle and storage at 4° C., b) one freeze/thaw cycle and storage at 4° C. with 10 vol. % glycerol, c) one freeze/thaw cycle and storage at −20° C., d) one freeze/thaw cycle and storage at −20° C. with 10 vol. % glycerol, e) one freeze/thaw cycle and storage at −80° C., f) two freeze/thaw cycles and storage at −80° C., g) two freeze/thaw cycles and storage at −80° C. with 10 vol. % glycerol, and h) storage at −80° C. for 25 days.

The samples were removed from storage and used directly in the assays. The total assay volume was 50 μl, containing 40 μM FPP, 10 μM NADPH, 200 ng tSqS, 1 mg/ml BSA and 1 mM DTT in 50 μM Tris buffer at a pH of 7.5. The reaction mixtures were incubated for 30 minutes at 37° C.

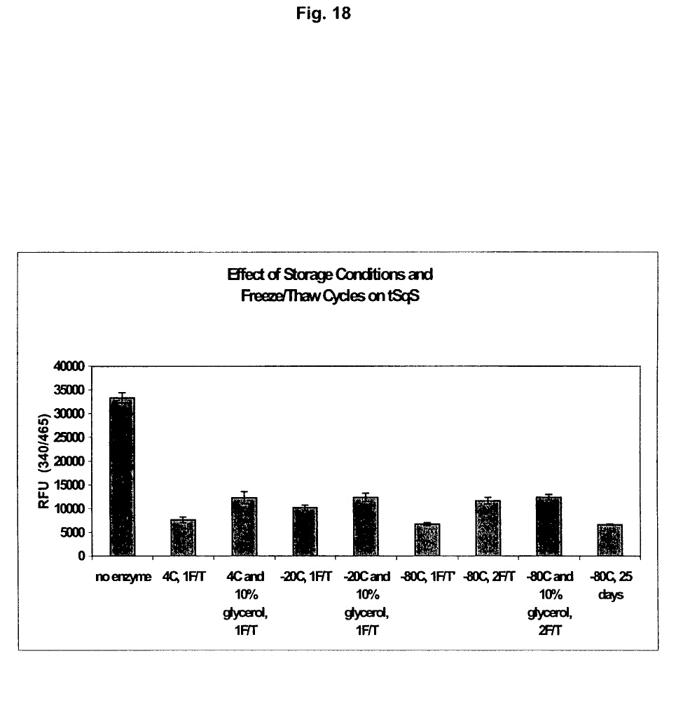

The data is shown in FIG. 18, where the values are the mean of triplicate values and the standard error is shown as error bars. Optimum results were obtained when the enzyme was stored at −80° C. without glycerol and only experiences one freeze-thaw cycle. However, leftover enzyme may be stored at 4° C. for up to 24 hours with no apparent deviation in activity.

### EXAMPLE 19

NADPH Stability at 4° C.

This experiment was performed to evaluate the stability of NADPH when stored at 4° C. 10 μM NADPH was stored at 4° C. for 24 hours. 40 μM substrate and assay buffer in total volume of 50 μM were incubated for 30 minutes at 37° C. in the absence of the enzyme (tSqS).

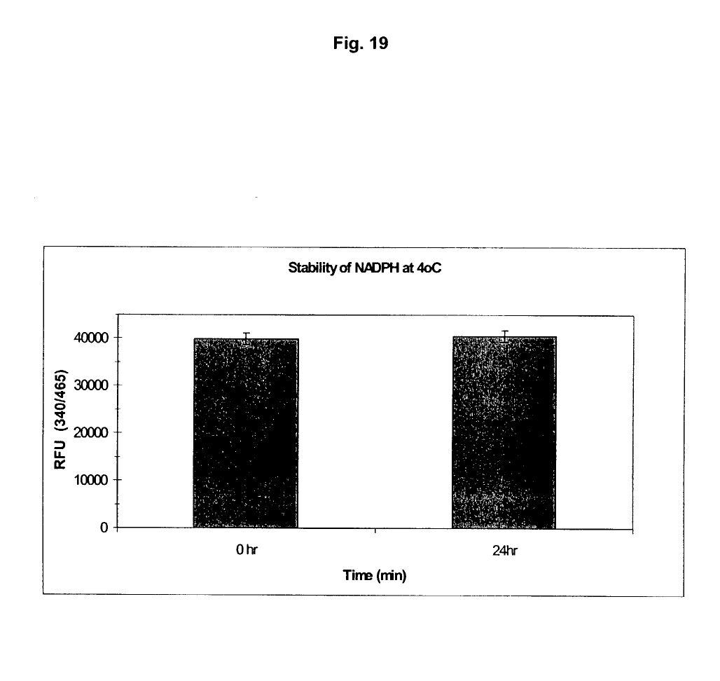

The results are shown in FIG. 19, which shows fluorescence (RFU) versus storage time, where the values are the mean of triplicate determinations with the standard error shown. The data show that virtually no deviation in fluorescence is observed when the NADPH is stored for 24 hours at 4° C.

### EXAMPLE 20

NADPH Stability at 37° C.

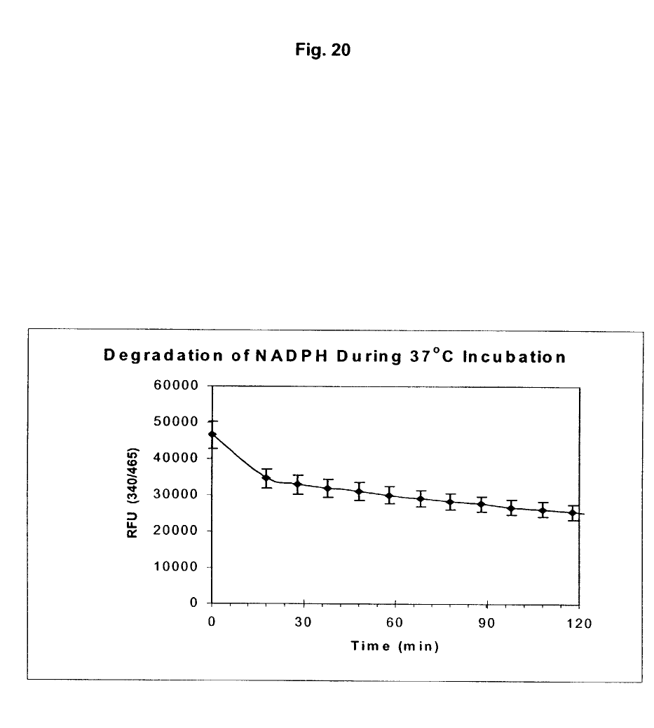

This experiment was performed to evaluate the stability of NADPH when incubated at 37° C. NADPH was incubated at 37° C. for 120 minutes in the absence of enzyme (tSqS) and the fluorescence was measured over time. The results are shown in FIG. 20, which show NADPH degrades over time at 37° C. (and would likely in the presence of tSqS). At the 30 minute timepoint, a background of approximately 36,000 RFU can be observed at a gain of 50 on the Tecan Ultra versus an initial value of about 45,000 RFU. The apparent degradation of NADPH is a temperature effect, especially at 37° C., and is detectable when the gain is set the same over time.

### EXAMPLE 21

DMSO Effects

DMSO may be present in the assay, particularly if it is added to the enzyme preparations when they are stored. This experiment was performed to evaluate the effect of DMSO on the assay results.

The reaction mixtures included 40 μM substrate, 10 μM NADPH, 0 or 125 ng enzyme and assay buffer in total volume of 50 μl. DMSO at various percentages (between 0 and 10 vol. %) was added to the reaction mixtures and they were allowed to incubate at 37° C. for 30 minutes.

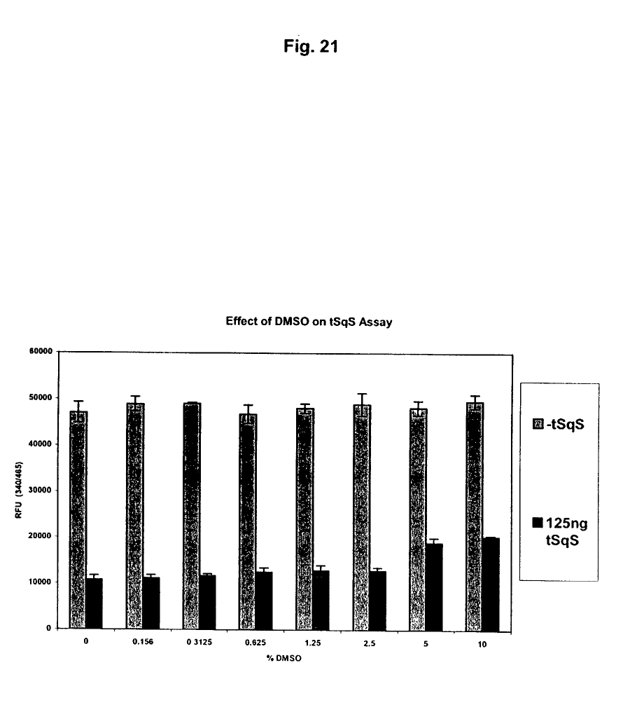

The results are shown in FIG. 21, where the values are the mean of triplicate determinations with standard error shown as error bars. The data indicate no significant effect of DMSO up to a concentration of 2.5%.

### EXAMPLE 22

Inhibition of tSqS Assay by EDTA

The conversion of the intermediate PSPP into squalene requires magnesium as a cofactor. This experiment was performed to show the inhibition of squalene synthase when the magnesium ion cofactor was chelated with EDTA.

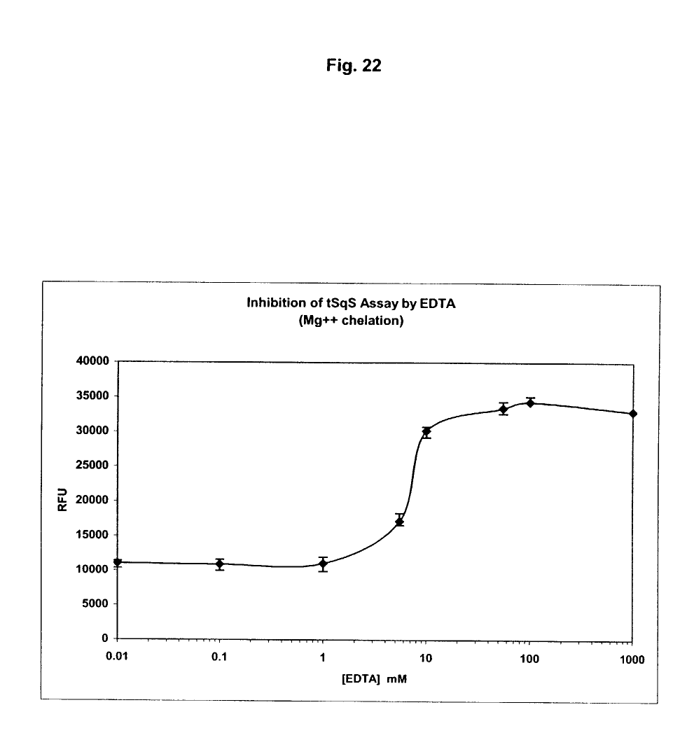

Various concentrations of EDTA were incubated for 30 minutes at 37° C. with 40 μM FPP, 10 μM NADPH, 10 mM Mg, 1 mg/ml BSA, 1 mM DTT, in 50 mM Tris buffer at a pH of 7.5. The results are shown in FIG. 22, where the values are the mean of triplicate determinations with standard error shown as error bars. The data shows that squalene synthase is inhibited via EDTA chelation of the magnesium ions. The IC₅₀ is approximately 8 mM, which is representative of approximately a 1:1 binding of the EDTA and the magnesium ions.

### EXAMPLE 23

384-well Statistics and Z-factor

This experiment was performed to confirm that the assay can be run in a high -throughput fashion. The SqS assay was tested for compatibility with the Bayer HTS system (although other high throughput systems can be used). The reagents were prepared just before addition via multidrop, and kept on ice throughout additions. The reactions were performed both with fresh reagents and reagents stored for 24 hours at 4° C. The assay was performed in an opaque white Greiner 384-well plate.

The total reaction volume (per well) was 50 μl, with 25 μl of 80 μM FPP and 20 μM of NADPH in assay buffer, and 25 μl enzyme diluted in assay buffer. The assay buffer without enzyme was added to one half of the plate, and reaction mixtures containing enzyme were added to the other. After an incubation of 30 minutes at 37° C., fluorescence was read at 340 nm excitation/465 nm emission using the TECAN Ultra. For the 24 hour time point, all reagents were stored at 4° C.

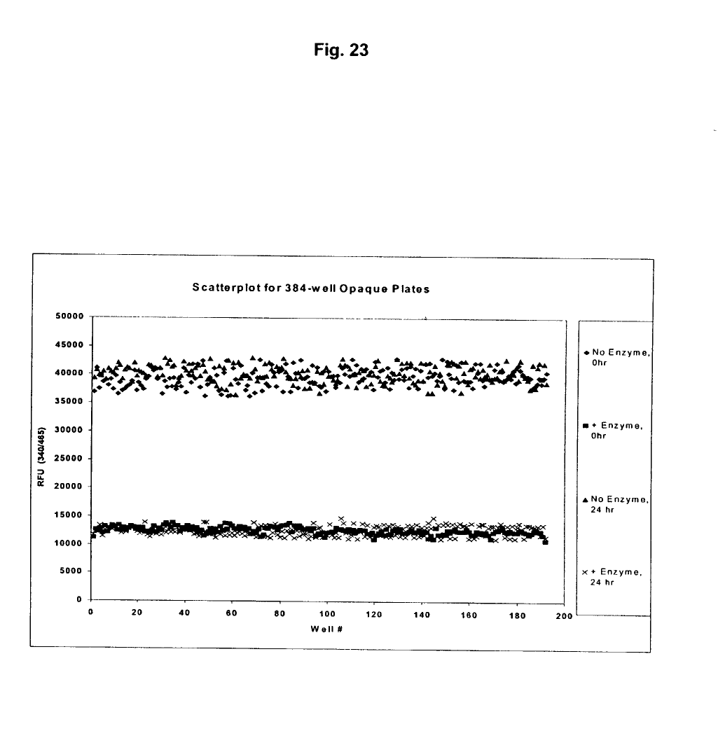

The results are shown in FIG. 23, which is a scatterplot of the 384-well plate in the presence and absence of the tSqS enzyme. The Z-factors were calculated to be 0.75 for the 0 hour time point, and 0.74 for the 24 hour time point. The data show that the assay is amenable to high throughput conditions.

### EXAMPLE 24

Comparison of Opaque vs. Solid Plates

This experiment was performed to compare two types of Greiner white plates, one with an opaque bottom and another with a clear bottom, to determine which gave the better Z-factors.

The assay was performed as in EXAMPLE 23. Opaque plates consistently yielded Z-factors of 0.7-0.75, while the clear bottom plates consistently yielded Z-factors of 0.60-0.65, explained by higher background and scatter. Accordingly, it may be preferred to use plates with an opaque bottom.

### EXAMPLE 25

Table of Plates Run for Z-factor Analysis.

384-well plates were used to determine the Z -factors using various conditions. The conditions included a) use of fresh reagents with a plate with an opaque bottom, b) use of reagents stored for 24 hours with a plate with an opaque bottom, c) use of reagents used directly from ice with a plate with an opaque bottom, d) use of reagents stored at room temperature in the tubing from the multidrop liquid handler for 20 minutes with a plate with an opaque bottom, e) use of fresh reagents with a clear bottom white plate, f) use of stored reagents with a clear bottom white plate, and g) the use of an opaque white plate with a different lot of tSqS.

The Plates were divided, with one half receiving enzyme and the other receiving assay buffer only. The data (not shown) showed that there was no significant difference in Z-factors in plates where reagents sat in RT tubing for up to 45 minutes.

### EXAMPLE 26

Squalene Synthase HTS Protocol

Based on the experiments in a number of examples discussed below, optimum conditions for performing high throughput assays for squalene synthase activity were determined. The optimum conditions are shown below, and the experiments that show how the optimum conditions were determined are shown in subsequent examples.

Final assay volume: 50 μl

All reagents are kept at 4° C.

Step 1

Dispense 25 μl of 2×Substrate/NADPH in Assay Buffer

Multidrop 1

Negative control- substrate, NADPH with no enzyme)

Step 2

Dispense 5 μl compound

TECAN Genesis

Step 3

Dispense 20 μl of enzyme in Assay Buffer

Multidrop 2

(Final concentration- 80 ng/well lot 030101)

Step 4

Incubate for 30 min at 37° C.

Step 5

Read Fluorescence at 340nm excitation/465nm emission

Gain set on no enzyme control.

Assay Stock Reagents

Assay Buffer

0.05M Tris pH 7.5

10 mM MgCl₂

1 mM DTT

1 mg/ml BSA

Substrate/NADPH (2×)

80 μM Farnesyl diphosphate (FPP) and

20 μM NADPH in Assay Buffer

Enzyme Stock (80 ng/20° μl lot 030101)

0.004 mg/ml in Assay Buffer Reagent List:

| | |
|---|---|
| Farnesyl Diphosphate (FPP) | Echelon (I-0150) |
| NADPH | Sigma (N-1630) |
| MgCl₂ | Sigma (M-2670) |
| BSA | Sigma (A-7906) |
| Tris HCl | Sigma (T-6666) |
| Dithiothreitol (DTT) | Sigma (D-5545) |

Assay Plate: Greiner solid white, non-tissue culture treated 384 well plate.

### SUMMARY

The Arabidopsis gene coding for truncated squalene synthase has been cloned into the Novagen pET30a vector and expressed in E. coli. The resulting protein was purified using a Ni/NTA affinity column. A fluorometric assay was developed for the indirect measurement of squalene. In the interest of assay sensitivity to inhibitors, the assay was performed at the approximate Km of FPP (40 μM). Statistical analysis using 125 ng truncated squalene synthase, 10 mM MgCl₂, 1 mM DTT, 1 mg/ml BSA, in 50 mM Tris pH 7.5, yielded a Z-factor of 0.75 and signal:background ratio of approximately 3.2. Data collected from experiments indicated that adding approximately 1 mg/ml BSA and using a relatively low concentration of NADPH were preferred for obtaining optimum results. The recommended concentration of the screening lot of enzyme was 0.004 mg/ml (80 ng/20 μl).

Initial assay development using a partially purified full-length gene was successful (data not shown). However, most protein still associated with the pelleted membranes regardless of extraction procedure. Thus, a higher amount of soluble enzyme would be necessary for the entire screen. The C-terminal hydrophobic region was therefore clipped off and the truncated squalene synthase re-evaluated in the assay.

To ensure a robust assay for the ultra HTS system, reagents were left to sit in multidrop tubing for up to 45 minutes. The result was no significant deviation in Z-factor.

The following non-limiting ranges of component concentrations are useful in performing the assay. The concentration of NADPH can range from about 0.0005 to about 0.5 mM. The concentration of FPP can range from about 0.001 to about 1 mM. The concentration of the magnesium ion cofactor can range from about 0.5 to 100 mM. Tris-HCl buffer can be present in concentrations of between about 10 and 100 mM at pH ranges between about 7.0 and 8.0.

While the foregoing describes certain embodiments of the invention, it will be understood by those skilled in the art that variations and modifications may be made and still fall within the scope of the invention.

1. A method for quantitating squalene formed by squalene synthase, comprising:
a) contacting NADPH, FPP and a magnesium ion cofactor with a squalene synthase;
b) exposing the reaction mixture to UV light; and
c) detecting the emitted fluorescent light,
wherein the decrease in the amount of fluorescent light is correlated to the amount of NADPH consumed in the synthesis of squalene, and
wherein the amount of squalene is correlated to the amount of NADPH consumed.

2. A method for determining squalene synthase activity, comprising:
a) contacting NADPH, FPP and a magnesium ion cofactor with a squalene synthase;
b) exposing the reaction mixture to UV light; and
c) detecting the emitted fluorescent light,
wherein the decrease in the amount of fluorescent light is correlated to the amount of NADPH consumed in the synthesis of squalene, and
wherein the amount of squalene is correlated to the amount of NADPH consumed.

3. The method of claim 2, wherein the activity of a squalene synthase is compared to a control.

4. The method of claim 2, wherein the squalene synthase is a plant squalene synthase.

5. The method of claim 4, wherein the plant squalene synthase is an Arabidopsis squalene synthase.

6. The method of claim 5, wherein Arabidopsis squalene synthase has the amino acid sequence of SEQ ID NO: 6.

7. The method of claim 5, wherein Arabidopsis squalene synthase is at least 80% identical to the amino acid sequence of SEQ ID NO: 6.

8. The method of claim 7, wherein Arabidopsis squalene synthase is at least 85% identical to the amino acid sequence of SEQ ID NO: 6.

9. The method of claim 8, wherein Arabidopsis squalene synthase is at least 90% identical to the amino acid sequence of SEQ ID NO: 6.

10. The method of claim 9, wherein Arabidopsis squalene synthase is at least 95% identical to the amino acid sequence of SEQ ID NO: 6.

11. The method of claim 2, wherein the wavelength of the UV light is approximately 330-350 nm and the wavelength of the fluorescent light emission is approximately 465 nm.

12. The method of claim 2, wherein the NADPH is present in the reaction mixture at an initial concentration of 0.0005 mM to 0.5 mM.

13. The method of claim 2, wherein the magnesium ion cofactor is present in said reaction mixture at an initial concentration of 0.5 mM to 100 mM.

14. The method of claim 2, wherein the FPP is present in the reaction mixture at an initial concentration of 0.001 mM to 1 mM.

15. The method of claim 2, wherein said reaction mixture further comprises 75-150 mM phosphate buffer at a pH of 7.0-8.0.

16. A method for identifying a test compound as an inhibitor or promoter of squalene synthase, comprising:
a) contacting NADPH, FPP and a magnesium ion cofactor with a squalene synthase in the presence and in the absence of a test compound;
b) exposing the reaction mixture to UV light; and
c) detecting the emitted fluorescent light over time,
wherein the decrease in the amount of fluorescent light over time is correlated to the amount of NADPH consumed in the synthesis of squalene, and
wherein the amount of squalene produced over time is correlated to the amount of NADPH consumed, and
wherein an increase in the amount of fluorescent light emission over time in the presence of the test compound indicates that the test compound is a squalene synthase inhibitor, and
wherein a decrease in the amount of fluorescent light emission over time in the presence of the test compound indicates that the test compound is a squalene synthase promoter.

17. The method of claim 16, wherein the squalene synthase is a human squalene synthase.

18. The method of claim 16, wherein the squalene synthase is a fungal squalene synthase.

19. The method of claim 16, wherein the squalene synthase is a plant squalene synthase.

20. The method of claim 19, wherein the plant squalene synthase is an Arabidopsis squalene synthase.

21. The method of claim 20, wherein the Arabidopsis squalene synthase has the amino acid sequence of SEQ ID NO: 6.

22. The method of claim 20, wherein the Arabidopsis squalene synthase is at least 90% identical to the amino acid sequence of SEQ ID NO: 6.

23. The method of claim 20, wherein the Arabidopsis squalene synthase is at least 95% identical to the amino acid sequence of SEQ ID NO: 6.

24. The method of claim 16, wherein the wavelength of the UV light is approximately 330-350 nm and the wavelength of the fluorescent light emission is approximately 465 nm.

25. The method of claim 16, wherein the NADPH is present in the reaction mixture at an initial concentration of 0.0005 mM to 0.5 mM.

26. The method of claim 16, wherein the magnesium ion cofactor is present in the reaction mixture at an initial concentration of 0.5 to 100 mM.

27. The method of claim 16, wherein the FPP is present in the reaction mixture at an initial concentration of 0.001 mM to 1 mM.

28. The method of claim 16, wherein the reaction mixture further comprises 10-100 mM Tris-HCl buffer at a pH of 7.0-8.0.

29. A method for identifying compounds capable of selectively promoting or inhibiting plant, fungal and/or animal squalene synthase activity, comprising:
a) combining FPP, NADPH, a magnesium ion cofactor and a plant squalene synthase to form a reaction mixture under conditions suitable for the production of squalene in the presence and absence of a test compound;
b) subjecting the reaction mixture to UV light and detecting fluorescent light emission over time,
c) determining the activity of the compound to promote or inhibit squalene synthase based on the fluorescent light emission over time,
d) repeating steps a-c using a fungal or animal squalene synthase, and
f) identifying compounds that selectively inhibit plant, fungal or animal squalene synthase.

30. The method of claim 29, wherein the squalene synthase is a plant squalene synthase.

31. The method of claim 30, wherein the plant squalene synthase is an Arabidopsis squalene synthase.

32. The method of claim 31, wherein the Arabidopsis squalene synthase has the amino acid sequence of SEQ ID NO: 6.

33. The method of claim 31, wherein the Arabidopsis squalene synthase is at least 90% identical to the amino acid sequence of SEQ ID NO: 6.

34. The method of claim 31, wherein the Arabidopsis squalene synthase is at least 95% identical to the amino acid sequence of SEQ ID NO: 6.

35. The method of claim 29, wherein the wavelength of the UV light is approximately 330-350 nm and the wavelength of the fluorescent light emission is approximately 465 nm.

36. The method of claim 29, wherein the FPP is present in the reaction mixture at an initial concentration of 0.0001 mM to 1 mM.

37. The method of claim 29, wherein the NADPH is present in the reaction mixture at an initial concentration of 0.0005 mM to 0.5 mM.

38. The method of claim 29, wherein the magnesium ion cofactor is present in the reaction mixture at an initial concentration of 0.5 mM to 100 mM.

39. The method of claim 29, wherein the reaction mixture further comprises 10-100 mM Tris-HCl buffer at a pH of 7.0-8.0.

40. The truncated squalene synthase having the sequence of SEQ ID NO: 6 or SEQ ID NO: 6 with conservative substitutions.

41. A polypeptide having squalene synthase activity, wherein said polypeptide is at least 90% identical to the amino acid sequence of SEQ ID NO: 6.

42. The polypeptide of claim 41, wherein said polypeptide is at least 95% identical to the amino acid sequence of SEQ ID NO: 6.

43. The oligonucleotide sequence of SEQ ID NO: 5 or a degenerative variant thereof.

44. An isolated nucleic acid comprising a sequence that encodes squalene synthase comprising an amino acid sequence having at least 90% sequence identity with SEQ ID NO: 6.

45. The nucleic acid of claim 44, wherein the amino acid sequence has at least 95% identity with SEQ ID NO: 6.

46. The nucleic acid of claim 44, wherein the squalene synthase has at least 50% of the activity of the squalene synthase identified by SEQ ID NO: 6.

47. The nucleic acid of claim 44, wherein the squalene synthase has at least 60% of the activity of the squalene synthase identified by SEQ ID NO: 6.

48. The nucleic acid of claim 44, wherein the squalene synthase has at least 80% of the activity of the squalene synthase identified by SEQ ID NO: 6.

49. The nucleic acid of claim 44, wherein the squalene synthase has at least 90% of the activity of the squalene synthase identified by SEQ ID NO: 6.


SEQUENCE LISTING (13 sequences)

SEQ ID NO: 1 - DNA, 1599 bp, Arabidopsis thaliana
gcgtcgatccacatcgcaggtgagggttcctgcaatttatccctcgtggtctctgaatctcagatcgtcgtcaacgaatcctccattttctgaatcaaaattttctggaaacaatggggagcttggggacgatgctgagatatccggatgacatatatccgctcctgaagatgaaacgagcgattgagaaagcggagaagcagatccctcctgagccacactggggtttctgctattcgatgctccacaaggtttcccgaagcttttctctcgttattcagcaactcaacaccgagctccgtaacgccgtgtgtgtgttctacttggttctccgagctcttgatactgttgaggatgatactagcataccaactgatgaaaaggttcccatcctgatagcttttcaccggcacatatacgatactgattggcattattcatgtggtacgaaggagtacaagattctaatggaccaatttcaccatgtttctgcagcttttttggaacttgaaaaagggtatcaagaggctatcgaggaaattactagaagaatgggtgcagggatggccaagtttatctgccaagaggtagaaactgttgatgactacgatgaatactgccactatgttgctgggcttgttggtttaggtttgtcgaaactcttcctcgctgcaggatcagaggttttgacaccagattgggaggcgatttccaattcaatgggtttatttctacagaaaacaaacattatcagagattatcttgaggacattaatgagataccaaaatcccgcatgttttggcctcgcgagatttggggcaaatatgctgacaagcttgaggatttaaaatacgaggagaacacaaacaaatccgtacagtgcttaaatgaaatggttaccaatgcgttgatgcatattgaagattgcctgaaatacatggtttccttgcgtgatccttccatatttcggttctgtgccatccctcagatcatggcgattggaacacttgcattatgctataacaatgaacaagtattcagaggcgttgtgaaactgaggcgaggtcttactgctaaagtcattgatcgtacaaagacaatggctgatgtctatggtgctttctatgatttttcctgcatgctgaagacaaaggttgacaagaacgatccaaatgccagtaagacactaaaccgacttgaagccgttcagaaactctgcagagacgctggagttcttcaaaacagaaaatcttatgttaatgacaaaggacaaccaaacagtgtctttattataatggttgtgattctactggccatagtctttgcatatctcagagcaaactgagtgatccatgtaagcgagtctgattgtatcaccatcattcaagatgttcagagcaaatttgagtgatgaagtaatctaggttgattcttattcacgccactgaatcctaagcaagattgtttccagaacaaacagagtttaagcatggtttagtctaaaaccatggattctattttagttactaccttcgttgtctaaacgtgcatttgttcatctatttttattccttgtgtttaaagttctttctttgttt

SEQ ID NO: 2 - protein, 410 aa, Arabidopsis thaliana
MGSLGTMLRYPDDIYPLLKMKRAIEKAEKQIPPEPHWGFCYSMLHKVSRSFSLVIQQLNTELRNAVCVFYLVLRALDTVEDDTSIPTDEKVPILIAFHRHIYDTDWHYSCGTKEYKILMDQFHHVSAAFLELEKGYQEAIEEITRRMGAGMAKFICQEVETVDDYDEYCHYVAGLVGLGLSKLFLAAGSEVLTPDWEAISNSMGLFLQKTNIIRDYLEDINEIPKSRMFWPREIWGKYADKLEDLKYEENTNKSVQCLNEMVTNALMHIEDCLKYMVSLRDPSIFRFCAIPQIMAIGTLALCYNNEQVFRGVVKLRRGLTAKVIDRTKTMADVYGAFYDFSCMLKTKVDKNDPNASKTLNRLEAVQKLCRDAGVLQNRKSYVNDKGQPNSVFIIMVVILLAIVFAYLRAN

SEQ ID NO: 3 - DNA, 69 bp, Arabidopsis thaliana
aacagtgtctttattataatggttgtgattctactggccatagtctttgcatatctcagagcaaactga

SEQ ID NO: 4 - protein, 22 aa, Arabidopsis thaliana
NSVFIIMVVILLAIVFAYLRAN

SEQ ID NO: 5 - DNA, 1164 bp, Arabidopsis thaliana
atggggagcttggggacgatgctgagatatccggatgacatatatccgctcctgaagatgaaacgagcgattgagaaagcggagaagcagatccctcctgagccacactggggtttctgctattcgatgctccacaaggtttcccgaagcttttctctcgttattcagcaactcaacaccgagctccgtaacgccgtgtgtgtgttctacttggttctccgagctcttgatactgttgaggatgatactagcataccaactgatgaaaaggttcccatcctgatagcttttcaccggcacatatacgatactgattggcattattcatgtggtacgaaggagtacaagattctaatggaccaatttcaccatgtttctgcagcttttttggaacttgaaaaagggtatcaagaggctatcgaggaaattactagaagaatgggtgcagggatggccaagtttatctgccaagaggtagaaactgttgatgactacgatgaatactgccactatgttgctgggcttgttggtttaggtttgtcgaaactcttcctcgctgcaggatcagaggttttgacaccagattgggaggcgatttccaattcaatgggtttatttctacagaaaacaaacattatcagagattatcttgaggacattaatgagataccaaaatcccgcatgttttggcctcgcgagatttggggcaaatatgctgacaagcttgaggatttaaaatacgaggagaacacaaacaaatccgtacagtgcttaaatgaaatggttaccaatgcgttgatgcatattgaagattgcctgaaatacatggtttccttgcgtgatccttccatatttcggttctgtgccatccctcagatcatggcgattggaacacttgcattatgctataacaatgaacaagtattcagaggcgttgtgaaactgaggcgaggtcttactgctaaagtcattgatcgtacaaagacaatggctgatgtctatggtgctttctatgatttttcctgcatgctgaagacaaaggttgacaagaacgatccaaatgccagtaagacactaaaccgacttgaagccgttcagaaactctgcagagacgctggagttcttcaaaacagaaaatcttatgttaatgacaaaggacaacca

SEQ ID NO: 6 - protein, 388 aa, Arabidopsis thaliana
MGSLGTMLRYPDDIYPLLKMKRAIEKAEKQIPPEPHWGFCYSMLHKVSRSFSLVIQQLNTELRNAVCVFYLVLRALDTVEDDTSIPTDEKVPILIAFHRHIYDTDWHYSCGTKEYKILMDQFHHVSAAFLELEKGYQEAIEEITRRMGAGMAKFICQEVETVDDYDEYCHYVAGLVGLGLSKLFLAAGSEVLTPDWEAISNSMGLFLQKTNIIRDYLEDINEIPKSRMFWPREIWGKYADKLEDLKYEENTNKSVQCLNEMVTNALMHIEDCLKYMVSLRDPSIFRFCAIPQIMAIGTLALCYNNEQVFRGVVKLRRGLTAKVIDRTKTMADVYGAFYDFSCMLKTKVDKNDPNASKTLNRLEAVQKLCRDAGVLQNRKSYVNDKGQP

SEQ ID NO: 7 - DNA, 31 bp, Arabidopsis thaliana
ggaattctcatggttgtcctttgtcattaac

SEQ ID NO: 8 - DNA, 28 bp, Arabidopsis thaliana
cgggatccatggggagcttggggacgat

SEQ ID NO: 9 - DNA, 31 bp, Arabidopsis thaliana; identical to SEQ ID NO: 7

SEQ ID NO: 10 - DNA, 150 bp, Arabidopsis thaliana
atgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatcc

SEQ ID NO: 11 - protein, 50 aa, Arabidopsis thaliana
MHHHHHHSSGLVPRGSGMKETAAAKFERQHMDSPDLGTDDDDKAMADIGS

SEQ ID NO: 12 - DNA, 1317 bp, Arabidopsis thaliana
atgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggggagcttggggacgatgctgagatatccggatgacatatatccgctcctgaagatgaaacgagcgattgagaaagcggagaagcagatccctcctgagccacactggggtttctgctattcgatgctccacaaggtttcccgaagcttttctctcgttattcagcaactcaacaccgagctccgtaacgccgtgtgtgtgttctacttggttctccgagctcttgatactgttgaggatgatactagcataccaactgatgaaaaggttcccatcctgatagcttttcaccggcacatatacgatactgattggcattattcatgtggtacgaaggagtacaagattctaatggaccaatttcaccatgtttctgcagcttttttggaacttgaaaaagggtatcaagaggctatcgaggaaattactagaagaatgggtgcagggatggccaagtttatctgccaagaggtagaaactgttgatgactacgatgaatactgccactatgttgctgggcttgttggtttaggtttgtcgaaactcttcctcgctgcaggatcagaggttttgacaccagattgggaggcgatttccaattcaatgggtttatttctacagaaaacaaacattatcagagattatcttgaggacattaatgagataccaaaatcccgcatgttttggcctcgcgagatttggggcaaatatgctgacaagcttgaggatttaaaatacgaggagaacacaaacaaatccgtacagtgcttaaatgaaatggttaccaatgcgttgatgcatattgaagattgcctgaaatacatggtttccttgcgtgatccttccatatttcggttctgtgccatccctcagatcatggcgattggaacacttgcattatgctataacaatgaacaagtattcagaggcgttgtgaaactgaggcgaggtcttactgctaaagtcattgatcgtacaaagacaatggctgatgtctatggtgctttctatgatttttcctgcatgctgaagacaaaggttgacaagaacgatccaaatgccagtaagacactaaaccgacttgaagccgttcagaaactctgcagagacgctggagttcttcaaaacagaaaatcttatgttaatgacaaaggacaaccatga

SEQ ID NO: 13 - protein, 80 aa, Arabidopsis thaliana
MHHHHHHSSGLVPRGSGMKETAAAKFERQHMDSPDLGTDDDDKAMADIGSMGSLGTMLRYPDDIYPLLKMKRAIEKAEKQ